(19) United States
(12) Patent Application Publication
(10) Pub. No.: US 2022/0040253 A1
(43) Pub. Date: Feb. 10, 2022

(54) Agonists of Human Kisspeptin Receptor for Modulating Sexual Desire

(71) Applicant: Université de Liège, Liège (BE)

(72) Inventors: Julie Bakker, Liège (BE); Sonia Ouerdi, Liège (BE)

(21) Appl. No.: 17/422,049

(22) PCT Filed: Jan. 24, 2019
(86) PCT No.: PCT/EP2019/051795

§ 371 Date: Jul. 9, 2021

(51) Int. Cl. A61K 38/09; A61P 15/00; A61K 31/137; A61K 31/496; A61K 31/568; A61K 31/5685; A61K 38/12; A61K 31/506; A61K 31/519; A61K 31/40; A61K 38/08

(52) U.S. Cl. CPC A61K 38/09; A61P 15/00; A61K 31/137; A61K 31/496; A61K 31/568; A61K 38/08; A61K 38/12; A61K 31/506; A61K 31/519; A61K 31/40; A61K 31/5685

(57) ABSTRACT

The present application relates to an agonist of a human kisspeptin receptor for use in a method of treating a disorder of sexual desire in human females, a method of treating a disorder of sexual desire in a human female patient, comprising administering to said patient a therapeutically effective amount of an agonist of a human kisspeptin receptor and a non-therapeutic method of enhancing libido or inducing sexual arousal in a human female subject, comprising administering to said subject an amount of an agonist of a human kisspeptin receptor sufficient to enhance libido in said subject.

### FIELD

The invention is broadly in the medical and lifestyle improvement fields, and more precisely relates to the treatment of disorders of sexual desire and non-therapeutic methods of enhancing libido in women.

### BACKGROUND

Sexual desire in women serves reproductive and recreational functions. Many women suffer from a low libido, sexual dysfunction, or low sexual desire, including hypoactive sexual desire disorder (HSDD), which represents the most severe form of low sexual desire. These conditions might lead to significant distress or interpersonal difficulty. Causes of decrease of sexual desire and function in women are multifactorial and include age, stress and anxiety, depression, mental disorders, diseases (diabetes), obesity, high blood pressure, high cholesterol, medication side effects, and low dopamine levels in the brain.

At present, there is no good treatment available for low sex drive, including HSDD, in women. Testosterone has been used in post-menopausal women, but also has strong adverse effects leading to virilization. On the other hand, flibanserin is used in women who have not gone through menopause to increase low libido. However, flibanserin only has a very limited effect and also has many uncomfortable and even dangerous side effects, such as increased occurrence of dizziness, sleepiness and nausea.

Accordingly, there remains a need for effective treatments for low libido and sexual desire in women.

### SUMMARY

The invention is at least in part based on the discovery that agonists of kisspeptin receptor enhance female libido or induce female sexual arousal and are further useful for the treatment of disorders of sexual desire in human females, including inter alia hypoactive sexual desire disorder (HSDD).

Accordingly, an aspect provides an agonist of human kisspeptin receptor for use in a method of treating a disorder of sexual desire in human females. A further aspect provides a method of treating a disorder of sexual desire in a human female patient, comprising administering to said patient a therapeutically effective amount of an agonist of human kisspeptin receptor. A further aspect provides a non-therapeutic method of enhancing libido in a human female subject, comprising administering to said subject an amount of an agonist of human kisspeptin receptor sufficient to enhance libido in said subject.

A further aspect provides a non-therapeutic method of inducing sexual arousal in a human female subject, comprising administering to said subject an amount of an agonist of human kisspeptin receptor sufficient to induce sexual arousal in said subject.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1. RP3V kisspeptin neurons are part of a motivational circuit triggered by male olfactory cues. (a) Removal of the vomeronasal organ (VNOx) but not ablation of the main olfactory epithelium by intranasal infusion of a zinc sulfate solution (ZnSO₄) with the vomeronasal organ intact (VNOi) disrupted male-odor induced activation of RP3V kisspeptin neurons as determined by the percentage of Fos/Kp colabeled cells. ***P≤0.001; Dunn's multiple comparison test; n=28/9/7/8/9. (b) Kisspeptin knockout (Kiss^{-/-}) female mice do not show a male-directed preference whereas female control littermates displayed a preference for the male. Strikingly, a single peripheral injection with kisspeptin (Kp-10) at a dose of 0.52 μg kg⁻¹ induced a very strong preference for the male in Kiss^{-/-} female mice. **P≤0.01; ***P≤0.001; One-sample t test (H0: mean equals 0); n=9 per group. (c) Stereotaxic injection with an AAV encoding a Cre-dependent caspase bilaterally into the RP3V led to a ˜70% decrease in the number of RP3V kisspeptin (Kp) cells in Cre⁺ compared to Cre⁻ females. ***P≤0.001; Unpaired t test; n=7. (d) Photomicrographs showing viral ablation of RP3V kisspeptin neurons in KissIC mice (left: Cre⁻; right Cre⁺). (e) Viral ablation of RP3V kisspeptin cells disrupted male-directed preferences in KissIC mice (Cre⁺) whereas a peripheral injection with Kp-10 induced a male-directed preference. *P≤0.05; ***P≤0.001; One-sample t test (H0: mean equals 0); **P≤0.01; Tukey's multiple comparison test; n=7 per group. Scale bar represents 100 μm. Bars represent the mean±SEM. For all experimental details, see Table 1.

FIG. 2. Kisspeptin expression in the arcuate nucleus is unaffected by viral ablation of the RP3V kisspeptin population. (a) Photomicrographs showing examples of kisspeptin-immunoreactivity (indicated by the arrows) in a KissIC (Cre⁻, left) and KissIC (Cre⁺, right) mouse; (b) Total amount of kisspeptin (Kp)-immunoreactivity indicated as total area (μm²) covered by thresholded pixels. Unpaired two-tailed t test; P=0.81; n=7 per genotype; Scale bar represents 100 μm. Bars represent means±SEM. For all experimental details, see Table 1.

FIG. 3. Fos activation upon mating in hypothalamic regions implicated in sexual behavior. Ovary intact, female mice were either mated with a sexually active male for 15 min or left alone in their homecage. Brains were processed for Fos immunoreactivity. Abbreviations are as follows: RP3V rostral periventricular area of the third ventricle of the hypothalamus; MPOA, medial preoptic area; ARC, arcuate nucleus; VMHv1, ventrolateral part of the ventromedial hypothalamus; MeA, medial amygdala. Bars represent means±SEM. Tukey's multiple comparison test; *P≤0.05, ***P≤0.001 compared to unmated; n=5. For all experimental details, see Table 1.

FIG. 4. RP3V kisspeptin neurons are an important component of the neural network regulating lordosis behavior. (a) Mating specifically activated kisspeptin neurons in the RP3V of ovary intact female mice as determined by the percentage of Fos/Kp colabeled cells. *P≤0.05; Unpaired t test; n=3/5. (b) A peripheral injection of kisspeptin-10 (Kp-10) at a dose of 0.52 μg/kg stimulated lordosis behavior. ***P≤0.001; Paired t test; n=8 per group. (c) Lordosis behavior is attenuated in kisspeptin knockout (Kiss^{-/-}) mice. Please note that the background strain of Kiss^{+/+} and Kiss^{-/-} mice is 129SvJ which showed overall lower levels of lordosis behavior compared to C57B16/j mice. *P≤0.05; Mann Whitney U test; n=7/10. (d) A peripheral injection with Kp-10 induced lordosis behavior in Kiss^{-/-} females. *P≤0.05; Paired t test; n=9. (e-f) Stereotaxic injection with an AAV encoding a Cre-dependent caspase bilaterally into the RP3V decreased lordosis behavior, but was restored by a peripheral Kp-10 injection. (e) **P≤0.01; Unpaired t test; *P≤0.05, (f) Paired t test; n=7 per group. (g) Anatomical drawing showing the position of the bilateral cannula holding optical fibers with 45° oriented mirrors tip into the RP3V. (h). Blue light photostimulation (10 Hz, 473 nm) elicited robust firing of kisspeptin neurons in KissIC mice brain slices which were injected with an AAV encoding a Cre-dependent channelrhodopsin (AAV-ChR2) bilaterally into the RP3V. (i) Blue light photostimulation (Stim) increased the expression of lordosis behavior in KissIC mice which were injected with AAV-ChR2 bilaterally into the RP3V. *P≤0.05, Paired t test; n=8 per group (Kiss Cre⁻ and Kiss Cre⁺). Bars represent the mean±SEM. For all experimental details, see Table 1.

FIG. 5. Fos/kisspeptin double-labeling upon mating in the RP3V. Representative photomicrographs from an unmated female (left panel) and mated female (right panel). Inserts show higher magnification. Black arrow heads show double-labeled Fos (in blue)/kisspeptin (in brown) neurons. White arrow heads show single-labeled kisspeptin neurons detected in brown. Scale bar represents 100 μm and 10 μm, respectively. For all experimental details, see Table 1.

FIG. 6. An intracerebroventricular injection with kisspeptin (Kp-10; 10.4 ng/kg) stimulates lordosis behavior in WT female mice. Bars represent means±SEM and the number of animals for each experimental group is given in each bar. Unpaired t test; *P≤0.05; n=5 (saline) and 7 (Kp10). For all experimental details, see Table 1.

FIG. 7. Lordosis behavior depends on the accessory olfactory system in female mice. (a) Lordosis behavior was strongly disrupted upon VNO removal, but only slightly after ablation of the MOE by infusion with a zinc sulfate solution (ZnSO₄). (b) Mating failed to activate RP3V kisspeptin neurons when both peripheral olfactory sensory input organs (VNOx/ZnSO₄) were ablated. Sham procedures were performed for each intervention as controls. No significant differences between control animals were found; therefore, all controls were combined into a single group. Bars represent the mean±SEM. *P≤0.05; **P≤0.01; ***P≤0.001; Dunn's multiple comparison test; (a) n=8/9/8/7; (b) n=8/9/8/7. For all experimental details, see Table 1.

FIG. 8. VNO removal (VNOx) or ablation of the MOE by intranasal infusion with a zinc sulfate (ZnSO₄) solution did not affect the number of mounts received from the stimulus male. Bars represent means±SEM. Dunn's multiple comparison test; P>0.99 for each group compared to Saline/VNOi group; n=7/8/7/6. For all experimental details, see Table 1.

FIG. 9. Mate preference but not lordosis behavior depends on GnRH signaling. (a) Genetic disruption of Dicer in GnRH neurons abolishing GnRH expression in GnRH:: Cre; Dicer^{loxP/loxP} mouse model induced a female-instead of a male-directed preference whereas control littermates showed a preference for the male (saline condition). A single peripheral GnRH injection at a dose of 0.025 mg kg⁻¹ induced a male-directed preference in GnRH:: Cre; Dicer^{loxP/loxP} female mice whereas a peripheral injection with kisspeptin (Kp-10) was not successful. *P≤0.05; One-sample t-test (H0: mean equals 0); n=6 (DicerloxP/loxP) or 8 (GnRH::Cre; DicerloxP/loxP). (b) Strikingly, such disruption of GnRH expression in GnRH:: Cre; Dicer^{loxP/loxP} mouse model did not affect lordosis behavior. Mann-Whitney U test; P=0.79; n=7/8. (c) A single injection with GnRH failed to stimulate lordosis behavior in Kiss^{-/-} mice. Paired t test; P=0.65; n=10. Bars represent the mean±SEM. For all experimental details, see Table 1.

FIG. 10. VMHv1 nNOS neurons are connected to RP3V kisspeptin neurons (a) mCherry-immunoreactive projections were detected in the VMHv1 after injection of AAV-ChR2 into the RP3V of KissIC/R26-BIZ mice (scale bar=50 μm); n=3 (b-d) Transsynaptic tracing reveals that nNOS neurons in the VMHv1 are (either directly or indirectly) connected to RP3V kisspeptin neurons (scale bar=50 μm). BL+ nNOS neurons are indicated with arrows. (e-g) Zoomed in image of insert shown in (d) (scale bar=10 μm). (h) The number of neurons expressing nNOS in the VMHv1, the number of BL+ cells and the overall percentage of BL+ nNOS neurons were not found to be significantly different (Bonferroni's Multiple Comparison test) between proestrus and metestrus/diestrus. n=6 for proestrus, n=8 for metestrus/diestrus Bars represent the mean±SEM. For all experimental details, see Table 1.

FIG. 11. Mate preference and lordosis behavior depend on nitric oxide signaling. (a) nNOS knockout (nNOS^{-/-}) female mice do not show a male-directed preference whereas female control littermates displayed a preference for the male. Strikingly, a single peripheral injection with SNAP+BAY 41-2272, NO donor and soluble guanylyl cyclase agonist induced a significant preference for the male in nNOS^{-/-} female mice. *P≤0.05; **P≤0.01; One-sample t test (H0: mean equals 0); n=6 (nNOS^{+/+}) and 7 (nNOS^{-/-}). (b) Lordosis behavior is disrupted in nNOS knockout (nNOS^{-/-}) mice, but restored by a peripheral injection of the NO donor SNAP (together with BAY 41-2272); *P≤0.05; **P≤0.01; Tukey's multiple comparison test; n=7 per group. (c) By contrast, a peripheral injection of either kisspeptin or GnRH failed to restore lordosis behavior in nNOS^{-/-} mice; ANOVA; n=7 per group. (d). A peripheral injection with SNAP+BAY 41-2272 induced lordosis behavior in Kiss^{-/-} mice; *P≤0.05; Paired t test; n=10 per group. Abbreviations: nNOS, the neuronal form of nitric oxide synthase; Kp-10, kisspeptin; SNAP, S-nitroso-N-acetylpenicillamine. Bars represent the mean±SEM. For all experimental details, see Table 1.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

A first aspect provides an agonist of human kisspeptin receptor for use in a method of treating a disorder of sexual desire in human females.

A related aspect provides a method of treating a disorder of sexual desire in a human female patient, comprising administering to said patient a therapeutically effective amount of an agonist of human kisspeptin receptor.

The human kisspeptin receptor may be any human receptor responsive to kisspeptin. In preferred embodiments, the human kisspeptin receptor is KISS1R and the agonist of human kisspeptin receptor is a KISS1R agonist. By means of additional guidance, human KISS1R is also known in the art as AXOR12, G-protein coupled receptor 54 (GPR54), hypogonadotropin-1 or metastin receptor. By means of additional guidance, human KISS1R mRNA is annotated under NCBI Genbank accession numbers NM_032551.4 (nucleotides 162 (start codon) to 1358 (stop codon) of NM_032551.4 constitute the KISS1R coding sequence). Human KISS1R protein sequence is annotated under NCBI Genbank accession numbers NP_115940.2 and Uniprot accession number Q969F8 the KISS1R protein sequence annotated as NP_115940.2 being reproduced below (SEQ ID NO: 1):

>NP_115940.2 kiSS-1 receptor [Homo sapiens]
MHTVATSGPNASWGAPANASGCPGCGANASDGPVPSPRAVDAWLVPLFFA
ALMLLGLVGNSLVIYVICRHKPMRTVTNFYIANLAATDVTFLLCCVPFTA
LLYPLPGWVLGDFMCKFVNYIQQVSVQATCATLTAMSVDRWYVTVFPLRA
LHRRTPRLALAVSLSIWVGSAAVSAPVLALHRLSPGPRAYCSEAFPSRAL
ERAFALYNLLALYLLPLLATCACYAAMLRHLGRVAVRPAPADSALQGQVL
AERAGAVRAKVSRLVAAVVLLFAACWGPIQLFLVLQALGPAGSWHPRSYA
AYALKTWAHCMSYSNSALNPLLYAFLGSHFRQAFRRVCPCAPRRPRRPRR
PGPSDPAAPHAELLRLGSHPAPARAQKPGSSGLAARGLCVLGEDNAPL

A skilled person can appreciate that any sequences represented in sequence databases or in the present specification may be of precursors of the respective proteins, polypeptides, peptides or nucleic acids and may include parts which are processed away from mature molecules. The term “agonist” broadly refers to an agent that is capable of binding to a receptor and activating the receptor to produce a biological response, such as activation or induction of an intracellular signalling mechanism or pathway in a cell. Without limitation, an agonist may be a naturally-occurring, semi-synthetic, synthetic or recombinantly produced substance. The binding of an agonist to a receptor may be reversible or irreversible.

In particular embodiments, the kisspeptin receptor agonist as intended herein is capable of binding to human kisspeptin receptor and/or activating human kisspeptin receptor-mediated signalling. For example, the kisspeptin receptor agonist may be capable of binding to human kisspeptin receptor when presented on the cell membrane of a eukaryotic cell, preferably an animal cell, more preferably a mammalian cell, even more preferably a human cell. For example, the kisspeptin receptor agonist may be capable of binding to native human kisspeptin receptor presented on the cell membrane of a human cell (such as a human neuron) endogenously expressing human kisspeptin receptor. For example, the kisspeptin receptor agonist may be capable of binding to human kisspeptin receptor presented on the cell membrane of a non-human cell, preferably a non-human eukaryotic cell, more preferably a non-human mammalian cell, heterologously or recombinantly expressing human kisspeptin receptor. For example, the kisspeptin receptor agonist may be capable of binding to native human kisspeptin receptor presented on the cell membrane of a human cell (such as a human neuron) endogenously expressing human kisspeptin receptor and of activating endogenous kisspeptin receptor-mediated signalling in the cell. A skilled person can appreciate that such cells can be in vivo (e.g., in a human, or in an animal model) or in vitro (e.g., in a tissue explant or in cell culture).

The ability to activate kisspeptin receptor-mediated signalling refers to the ability of the agonist to mimic, reproduce or approximate the signal transduction effect and/or activity of natural kisspeptin binding to the kisspeptin receptor on a human cell containing the components of the intracellular signalling pathway downstream of the kisspeptin receptor. Activation of kisspeptin receptor-mediated signalling may be suitably determined and/or quantitated by measuring the secretion of gonadotrophins, such as luteinizing hormone (LH). This may be achieved by any methods known in the art for determining and/or quantifying the secretion of gonadotrophins, such as by radioimmunoassay.

In particular embodiments, the agonist as intended herein may be considered capable of activating kisspeptin receptor-mediated signalling if an experimentally meaningful amount of the agonist (for example but without limitation, an amount equimolar to an amount of kisspeptin known to activate kisspeptin receptor-mediated signalling) enhances kisspeptin receptor-mediated signalling—for example, enhances said signalling at least 5-fold more, at least 10-fold more, at least 20-fold more, at least 30-fold more, at least 40-fold more, at least 50-fold more, at least 100-fold more, at least 250-fold more, at least 500-fold more, at least 750-fold more, at least 1000-fold more, at least 1×10⁴-fold more, or at least 1×10⁵-fold more—compared to kisspeptin receptor-mediated signalling baseline or background induced by a comparable experimentally meaningful amount of a substance known to have no effect on kisspeptin receptor-mediated signaling (i.e., a neutral substance or negative control).

The terms “bind”, “interact”, “specifically bind” or “specifically interact” as used throughout this specification mean that an agonist binds to or influences one or more desired molecules or substances substantially to the exclusion of other molecules or substances which are random or unrelated, and optionally substantially to the exclusion of other molecules that are structurally related. The terms do not necessarily require that an agonist binds exclusively to its intended target(s). For example, an agonist may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, such as, e.g., at least about 1000-fold or more greater, at least about 1×10⁴-fold or more greater, or at least about 1×10⁵-fold or more greater, than its affinity for a non-target molecule.

The binding or interaction between the agonist and its intended target(s) may be covalent (i.e., mediated by one or more chemical bonds that involve the sharing of electron pairs between atoms) or, more typically, non-covalent (i.e., mediated by non-covalent forces, such as for example, hydrogen bridges, dipolar interactions, van der Waals interactions, and the like). Preferably, the agonist may bind to or interact with its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹, wherein K_{A}=[A_T]/[A][T], A denotes the agonist, T denotes the intended target. Determination of K_{A} can be carried out by methods known in the art, such as for example, using equilibrium dialysis and Scatchard plot analysis. The binding of an agonist as described herein to a target and the affinity and specificity of said binding may be determined by any methods known in the art. Non-limiting examples thereof include co-immunoprecipitation, bimolecular fluorescence complementation, affinity electrophoresis, label transfer, phage display, proximity ligation assay (PLA), Tandem affinity purification (TAP), in-silico docking and calculation of the predicted Gibbs binding energy and competition binding assays.

In particular embodiments, the kisspeptin receptor agonist as intended herein is capable of binding to the extracellular N-terminal domain and/or one or more (one, two, or all three) extracellular loops of the kisspeptin receptor. Preferably, the kisspeptin receptor agonist as intended herein is capable of binding to the extracellular N-terminal domain and/or the first or second extracellular loop of the kisspeptin receptor.

In preferred embodiments, the kisspeptin receptor agonist as intended herein is capable of binding to the extracellular N-terminal domain and/or one or more (one, two, or all three) extracellular loops of KISS1R. Preferably, the kisspeptin receptor agonist as intended herein is capable of binding to the extracellular N-terminal domain and/or the first or second extracellular loop of KISS1R.

In particular embodiments, amino acids 1 to 46 of NP_115940.2 constitute the extracellular N-terminal domain of human KISS1R, and amino acids 102 to 120, 179 to 205, 285 to 305 of NP_115940.2 constitute the first, the second and third extracellular loops of KISS1R, respectively. Accordingly, in particular embodiments, the kisspeptin receptor agonist, preferably the KISS1R agonist, as intended herein is capable of binding to amino acid sequence MHTVATSGPNASWGAPANASGCPGCGANASDGPVPSPRAVDAWLVP (SEQ ID NO: 2) and/or one or more of the amino acid sequences LYPLPGWVLGDFMCKFVNY (SEQ ID NO: 3), ALHRLSPGPRAYCSEAFPSRALERAFA (SEQ ID NO: 4), and LQALGPAGSWHPRSYAAYALK (SEQ ID NO: 5). Preferably, the KISS1R agonist, as intended herein is capable of binding to amino acid sequence SEQ ID NO: 2 and/or one or more of the amino acid sequences SEQ ID NO: 3 and SEQ ID NO: 4.

In particular embodiments, amino acids 1 to 41 of NP_115940.2 constitute the extracellular N-terminal domain of human KISS1R, and amino acids 102 to 119, 181 to 205, 287 to 304 of NP_115940.2 constitute the first, the second and third extracellular loops of KISS1R, respectively. Accordingly, in particular embodiments, the kisspeptin receptor agonist, preferably the KISS1R agonist, as intended herein is capable of binding to amino acid sequence MHTVATSGPNASWGAPANASGCPGCGANASDGPVPSPRAVDAW (SEQ ID NO: 6) and/or one or more of the amino acid sequences LYPLPGWVLGDFMCKFVN (SEQ ID NO: 7), HRLSPGPRAYCSEAFPSRALERAFA (SEQ ID NO: 8), and ALGPAGSWHPRSYAAYAL (SEQ ID NO: 9). Preferably, the KISS1R agonist, as intended herein is capable of binding to amino acid sequence SEQ ID NO: 6 and/or one or more of the amino acid sequences SEQ ID NO: 7 and SEQ ID NO: 8.

In other particular embodiments, amino acids 1 to 46 of NP_115940.2 constitute the extracellular N-terminal domain of human KISS1R, and amino acids 102 to 120, 179 to 202, 285 to 305 of NP_115940.2 constitute the first, the second and third extracellular loops of KISS1R, respectively.

Accordingly, in particular embodiments, the kisspeptin receptor agonist, preferably the KISS1R agonist, as intended herein is capable of binding to amino acid sequence MHTVATSGPNASWGAPANASGCPGCGANASDGPVPSPRAVDAWLVP (SEQ ID NO: 10) and/or one or more of the amino acid sequences LYPLPGWVLGDFMCKFVNY (SEQ ID NO: 11), ALHRLSPGPRAYCSEAFPSRALER (SEQ ID NO: 12), and LQALGPAGSWHPRSYAAYALK (SEQ ID NO: 13). Preferably, the KISS1R agonist, as intended herein is capable of binding to amino acid sequence SEQ ID NO: 10 and/or one or more of the amino acid sequences SEQ ID NO: 11 and SEQ ID NO: 12.

In particular embodiments, the kisspeptin receptor agonist comprises or is selected from a group consisting of a chemical substance, an antibody, an antibody fragment, an antibody-like protein scaffold, a protein or polypeptide, a peptide, a peptidomimetic, an aptamer, a photoaptamer, a spiegelmer and a nucleic acid.

The kisspeptin receptor agonist as intended herein may comprise a combination of two or more of a chemical substance, an antibody, an antibody fragment, an antibody-like protein scaffold, a protein or polypeptide, a peptide, a peptidomimetic, an aptamer, a photoaptamer, a spiegelmer and a nucleic acid. For example, the kisspeptin receptor agonist as intended herein may comprise a combination of one or more polypeptide regions and one or more non-polypeptide regions.

As used herein, the term “chemical substance” is used in its broadest sense and generally refers to any substantially pure substance that has a constant chemical composition and characteristic properties. The chemical substance may be an organic molecule, preferably a small organic molecule. The term “small molecule” refers to compounds, preferably organic compounds, with a size comparable to those organic molecules generally used in pharmaceuticals. The term excludes biological macromolecules (e.g., proteins, polypeptides, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “antibody” is used herein in its broadest sense and generally refers to any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The term antibody thereby includes a whole immunoglobulin molecule, a monoclonal antibody, a chimeric antibody, a humanized antibody, a human antibody, or an immunologically effective fragment of any of these. The term thus specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo. The term “antibody fragment” or “antigen-binding moiety” comprises a portion or region of a full length antibody, generally the antigen binding or variable domain thereof. Examples of antibody fragments include Fab, Fab′, F(ab)2, Fv, scFv fragments, single domain (sd)Fv, such as V_{H} domains, V_{L} domains and V_{HH} domains, diabodies, linear antibodies, single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning. In certain embodiments, the antibody fragment may be a Nanobody®.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. In particular, 5-methylcytosine substitutions have been shown to increase nucleic acid duplex stability and may be preferred base substitutions in for example antisense agents, even more particularly when combined with 2′-O-methoxyethyl sugar modifications. Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Nucleic acid molecules comprising at least one deoxyribonucleoside unit may be typically referred to as deoxyribonucleic acids or DNA. Such deoxyribonucleoside unit(s) comprise 2′-H. Nucleoside units may be linked to one another by any one of numerous known inter-nucleoside linkages, including inter alia phosphodiester linkages common in naturally-occurring nucleic acids. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof.

The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, e.g., produced natively or endogenously by a cell or a tissue and optionally isolated therefrom. A nucleic acid can be recombinant, i.e., produced by recombinant DNA technology, and/or can be, partly or entirely, chemically or biochemically synthesised. Without limitation, a nucleic acid can be produced recombinantly by a suitable host or host cell expression system and optionally isolated therefrom (e.g., a suitable bacterial, yeast, fungal, plant or animal host or host cell expression system), or produced recombinantly by cell-free transcription, or non-biological nucleic acid synthesis. A nucleic acid can be double-stranded, partly double stranded, or single-stranded. Where single-stranded, the nucleic acid can be the sense strand or the antisense strand. In addition, nucleic acid can be circular or linear.

In particular embodiments, the kisspeptin receptor agonist as intended herein comprises or is selected from a group consisting of a protein, polypeptide, or a peptide.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D and L stereoisomers, provided their structure allows such stereo-isomeric forms. Amino acids are referred to herein by either their name, their commonly known three letter symbols or by the one-letter symbols recommended by the IUPAC-IUB Biochemical Nomenclature Commission. The term “naturally occurring” generally refers to materials which are found in nature and are not manipulated by man. The terms “non-naturally occurring”, “un-natural” and the like generally refer to a material that is not found in nature or that has been structurally modified, semi-synthesised or synthesised by man. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins or polypeptides may be advantageous in a number of different ways. For example, D-amino acid-containing polypeptides exhibit increased stability in vitro or in vivo compared to L-amino acid-containing counterparts. More specifically, D-amino acid-containing polypeptides may be more resistant to endogenous peptidases and proteases, thereby providing improved bioavailability of the agent and prolonged lifetimes in vivo.

A protein, polypeptide or peptide can be naturally occurring, e.g., present in or isolated from nature, e.g., produced or expressed natively or endogenously by a cell or tissue and optionally isolated therefrom. A protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological protein, polypeptide or peptide synthesis.

The reference to any proteins, polypeptides, peptides or nucleic acids encompass such proteins, polypeptides, peptides or nucleic acids of any organism where found, and particularly of animals, preferably warm-blooded animals, more preferably vertebrates, yet more preferably mammals, including humans and non-human mammals, still more preferably of humans.

In particular embodiments, the kisspeptin receptor agonist comprises, consists essentially of or consists of kisspeptin or a biologically active fragment or variant of kisspeptin, or a pharmaceutically acceptable salt thereof.

In more particular embodiments, the kisspeptin is human kisspeptin, or a pharmaceutically acceptable salt thereof.

Typically, as used herein, the term “human kisspeptin” refers to native or wild-type human kisspeptin. This may particularly denote human kisspeptin peptides or polypeptides with native or wild-type amino acid sequence, i.e., ones of which the primary sequence is identical to that of human kisspeptin found in or isolated from nature. Hence, the qualifier “native” or “wild-type” in this connection relates to the structure, such as in particular the primary amino acid sequence, of the human kisspeptin peptides or polypeptides, rather than to their effective origin or source. For example, such human kisspeptin peptides or polypeptides may be isolated from human tissues or cells endogenously expressing human kisspeptin, or may be obtained by other means, such as by recombinant expression, cell-free translation, or non-biological peptide synthesis. By means of additional guidance, human kisspeptin is also known in the art as Kiss1, KISS1, KiSS1, or metastasis-supressor Kiss-1. KiSS-1 cDNA was initially isolated from malignant melanoma cells as a novel human malignant melanoma metastasis-suppressor gene by Lee J H et al. (J Natl Cancer Inst, 1996, 89(20): 1549). Kisspeptins comprise a family of peptides derived from the Kiss1 gene, which when translated yields a 138-amino-acid preproprotein. In vivo proteolytic cleavage of the preproprotein generates the active form of Kisspeptin, namely Kisspeptin-54 (also known as metastin). Kisspeptin-14 (also known as Kiss14 or Kp-14) and Kisspeptin-13 (also known as Kiss13 or Kp-13) have been isolated from the placenta and may be in vivo degradation products from Kisspeptin-54. The shorter kisspeptins all contain the same consecutive 10 amino-acids. A synthetic peptide containing only these 10 amino acids, namely Kisspeptin-10 (also known as Kiss10 or Kp-10), retains biological activity in vivo.

By means of an example, human KISS1 gene is annotated under NCBI Genbank (http://www.ncbi.nlm.nih.gov/) Gene ID 3814. Human KISS1 mRNA is annotated under NCBI Genbank accession number NM_002256.3. Nucleotides 155 (start codon) to 571 (stop codon) of NM_002256.3 constitute the KISS1 coding sequence. Human KISS1 preproprotein sequence is annotated under NCBI Genbank accession number NP_002247.3, and Uniprot (www.uniprot.org) accession number Q15726, and is further reproduced below (SEQ ID NO: 14):

>NP_002247.3 metastasis-suppressor KiSS-1
preproprotein [Homo sapiens]
MNSLVSWQLLLFLCATHFGEPLEKVASVGNSRPTGQQLESLGLLAPGEQS
LPCTERKPAATARLSRRGTSLSPPPESSGSPQQPGLSAPHSRQIPAPQGA
VLVQREKDLPNYNWNSFGLRFGKREAAPGNHGRSAGRG

A skilled person can appreciate that any sequences represented in sequence databases or in the present specification may be of precursors of the respective proteins, polypeptides, peptides or nucleic acids and may include parts which are processed away from mature molecules. For example, the human KISS1 preproprotein comprises the N-terminal peptide signal MNSLVSWQLLLFLCATHFG (SEQ ID NO: 15), which will be cleaved in vivo from the KISS1 preproprotein after completion of translocation to generate a free signal peptide and a KISS1 proprotein comprising an amino acid sequence as set forth in SEQ ID NO: 16:

EPLEKVASVGNSRPTGQQLESLGLLAPGEQSLPCTERKPAATARLSRRGT
SLSPPPESSGSPQQPGLSAPHSRQIPAPQGAVLVQREKDLPNYNWNSFGL
RFGKREAAPGNHGRSAGRG.

In particular embodiments, the kisspeptin receptor agonist comprises, consists essentially of or consists of human kisspeptin as set forth in SEQ ID NO: 16. In other embodiments, the agonist comprises, consists essentially of or consists of an amino acid sequence having at least 80%, at least 85%, at least 90%, at least 95%, or at least 99% sequence identity to the amino acid sequence SEQ ID NO: 16, or a pharmaceutically acceptable salt thereof. In preferred embodiments, the agonist comprises, consists essentially of or consists of an amino acid sequence having at least 90% sequence identity to the amino acid sequence SEQ ID NO: 16, or a pharmaceutically acceptable salt thereof.

The KISS1 proprotein is typically proteolysed in vivo to peptides of various lengths. Amino acids 68 to 121, 108 to 121, 109 to 121, or 112 to 121 of NP_002247.3 constitute, respectively, human kisspeptin-54, human kisspeptin-14, human kisspeptin-13, or human kisspeptin-10. The amino acid sequences of these human kisspeptin fragments is shown below:

human kisspeptin-54:
(SEQ ID NO: 17)
GTSLSPPPESSGSPQQPGLSAPHSRQIPAPQGAVLVQREKDLPNYNWNSF
GLRF;
human kisspeptin-14:
(SEQ ID NO: 18)
DLPNYNWNSFGLRF;
human kisspeptin-13:
(SEQ ID NO: 19)
LPNYNWNSFGLRF;
human kisspeptin-10:
(SEQ ID NO: 20)
YNWNSFGLRF.

The kisspeptins may be isolated or purified from a naturally occurring source of the protein, polypeptide, or peptide, or may be produced by any recombinant, semi-synthetic or synthetic means, or combinations of such available in the art, as described elsewhere herein.

In particular embodiments, the kisspeptin receptor agonist is selected from the group consisting of human kisspeptin-54, human kisspeptin-14, human kisspeptin-13, human kisspeptin-10, pharmaceutically acceptable salts thereof, and combinations thereof. In particular embodiments, the kisspeptin receptor agonist comprises, consists essentially of or consists of an amino acid sequence having at least 80%, at least 85%, at least 90%, at least 95%, or at least 99% sequence identity to the amino acid sequence SEQ ID NO: 17, SEQ ID NO: 18, SEQ ID NO: 19, or SEQ ID NO: 20.

For example, the agonist may be a synthetic kisspeptin selected from the group consisting of TAK-448 (CAS number: 1234319-68-6) and TAK-683 (CAS number: 872719-49-8), or a pharmaceutically acceptable salt thereof.

In particular embodiments, the kisspeptin receptor agonist is human kisspeptin-54 or a pharmaceutically acceptable salt thereof.

In particular embodiments, the kisspeptin receptor agonist is human kisspeptin-14 or a pharmaceutically acceptable salt thereof.

In particular embodiments, the kisspeptin receptor agonist is human kisspeptin-13 or a pharmaceutically acceptable salt thereof.

In further preferred embodiments, the kisspeptin receptor agonist is human kisspeptin-10 or a pharmaceutically acceptable salt thereof The term “fragment” as used throughout this specification with reference to a protein, polypeptide or peptide generally denotes a portion of the protein, polypeptide or peptide, such as typically an N- and/or C-terminally truncated form of the protein, polypeptide or peptide. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said protein, polypeptide or peptide. For example, insofar not exceeding the length of the full-length protein, polypeptide or peptide, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500, ≥600, ≥700, ≥800, ≥900 or ≥1000 consecutive amino acids of the corresponding full-length protein, polypeptide or peptide.

The terms encompass fragments arising by any mechanism, in vivo and/or in vitro, such as, without limitation, by alternative transcription or translation, exo- and/or endo-proteolysis, exo- and/or endo-nucleolysis, or degradation of the protein, polypeptide, peptide or nucleic acid, such as, for example, by physical, chemical and/or enzymatic proteolysis or nucleolysis. Preferably, the fragments are directly produced (i.e. without the need of fragmentation of the full-length protein, polypeptide or peptide) by any recombinant, semi-synthetic or synthetic means, or combinations of such available in the art. For example, production of the fragment of interest may be achieved by introducing a recombinant nucleic acid encoding the fragment operably linked to suitable regulatory sequences into a suitable cell or organism, bringing said fragment to expression in said cell or organism, and isolating the expressed fragment from said cell or organism and/or from the surrounding culture medium or supernatant.

In particular embodiments, the agonist comprises, consists essentially of or consists of at least about 30%, e.g., at least about 50% or at least about 70%, preferably at least about 80%, e.g., at least about 85%, more preferably at least about 90%, and yet more preferably at least about 95% or even about 99% contiguous amino acids of human kisspeptin, such as set forth in SEQ ID NO: 20, 16, 17, 18 or 19.

The term “variant” of a protein, polypeptide, or peptide broadly encompasses variants the amino acid sequence of which is the same as the amino acid sequence of said protein, polypeptide, or peptide, but which comprise one or more other modifications vis-à-vis said protein, polypeptide, or peptide; as well as variants which differ from said protein, polypeptide or peptide in their amino acid sequence; and combinations thereof.

A variant of a protein, polypeptide or peptide may comprise one or more chemically modified amino acid residues vis-à-vis said protein, polypeptide, or peptide. Non-limiting examples of chemical modifications of amino acid residues include acetylation, glycosylation, succinylation, phosphorylation, sulfonation, methylation, ubiquitination, formylation, biotinylation, amidation, cyclization, and fatty acid conjugation, but not limited thereto. Chemically modified amino acid residues modifications may be located N-terminally, internally or C-terminally of the protein, polypeptide, or peptide. For example, N-terminal acetylation of recombinant proteins, polypeptides or peptides may make the protein, polypeptide or peptide more closely mimic the charge state in the native protein, polypeptide or peptide. In addition, this modification may stabilize the resulting protein, polypeptide or peptide, and enhances its ability to resist enzymatic degradation by exopeptidases.

A variant of a protein, polypeptide, or peptide may be a fusion protein, polypeptide, or peptide, wherein the protein, polypeptide, or peptide is chemically conjugated, non-covalently bound, or translationally fused to one or more other proteins, polypeptides or peptides. Other proteins, polypeptides or peptides may include signal-generating compounds (e.g. enzyme or fluorophore), diagnostic or detectable markers (e.g. green fluorescent protein (GFP), or chloramphenicol acetyl transferase (CAT)), amino acid sequences used for purification of recombinant proteins, polypeptides or peptides (e.g. FLAG, polyhistidine (e.g., hexahistidine), hemagluttanin (HA), glutathione-S-transferase (GST), or maltose-binding protein (MBP)), signal sequences and amino acid sequences used to direct or enhance the transport of the protein, polypeptide or peptide to a target cell (e.g. blood-brain barrier shuttle peptides), but are not limited thereto. The amino acid sequence can be fused at the N-terminus and/or C-terminus of the agonist as intended herein, optionally by use of a spacer (e.g. aminohexanoic acid (Ahx) or poly(ethylene)glycol (PEG)).

In particular embodiments, said variant is a fusion protein of the kisspeptin receptor agonist as intended herein and an amino acid sequences used to direct or enhance the transport of the kisspeptin receptor to a target cell, preferably a blood-brain barrier shuttle peptide, as described elsewhere herein.

Variants which carry amino acid sequence variations vis-à-vis the recited protein, polypeptide, or peptide, such as, e.g., amino acid deletions, additions and/or substitutions typically have an amino acid sequence which is substantially identical (i.e., largely but not wholly identical) to the sequence of said protein, polypeptide, or peptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical to the sequence of the recited protein, polypeptide, or peptide. Preferably, a variant may display such degrees of identity to a recited protein, polypeptide, or peptide when the whole sequence of the recited protein, polypeptide, or peptide is queried in the sequence alignment (i.e., overall sequence identity). Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10). A variant of a protein, polypeptide, or peptide may be a homologue (e.g., orthologue or paralogue) of said protein, polypeptide, or peptide. As used herein, the term “homology” generally denotes structural similarity between two macromolecules from same or different taxons, wherein said similarity is due to shared ancestry.

A variant of a protein, polypeptide, or peptide may comprise one or more amino acid additions, deletions, or substitutions relative to (i.e., compared with) the corresponding protein, polypeptide or peptide. For example, a variant (substitution variant) of a protein, polypeptide, or peptide may comprise up to 70 (e.g., not more than one, two, three, four, five, six, seven, eight, nine, ten, 12, 15, 20, 25, 30, 35, 40, 50, 60, or 70) conservative amino acid substitutions relative to (i.e., compared with) the corresponding protein, polypeptide or peptide; and/or a variant (substitution variant) of a protein, polypeptide, or peptide may comprise up to 20 (e.g., not more than one, two, three, four, five, six, seven, eight, nine, ten, 11, 12, 13, 14, 15, 16, 17, 18, or 19) non-conservative amino acid substitutions relative to (i.e., compared with) the corresponding protein, polypeptide or peptide.

A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

Variants of proteins, polypeptides, or peptides also include proteins, polypeptides, or peptides in which one or more natural amino acid residues are substituted with non-natural (e.g. citrulline, ornithine, aminobenzoic acid, hydroxyproline, E-Acetyl-lysine, 3-amino-propionic acid, aminobenzoic acid, 6-aminocaproic acid, aminobutyric acid, mercaptopropionic acid, 3-nitro-tyrosine, norleucine, pyroglutamic acid or labeled amino acid residues, such as amino acid residues labelled with an isotope, fluorescein isothiocyanate (FITC), biotin), as well as proteins, polypeptides, or peptides in which L-amino acid residues are substituted with D-amino acid residues.

As described elsewhere herein, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the agonist and prolonged lifetimes in vivo.

Accordingly, in particular embodiments, said variant comprises one or more non-naturally occurring amino acids, chemically modified amino acids and/or D-amino acids. Alternatively or in addition, for example, a variant (deletion variant) of a protein, polypeptide, or peptide may lack up to 20 amino acid segments (e.g., one, two, three, four, five, six, seven, eight, nine, ten, 11, 12, 13, 14, 15, 16, 17, 18, 19, or 20 segments) relative to (i.e., compared with) the corresponding protein, polypeptide or peptide. The deletion segment(s) may each independently consist of one amino acid, two contiguous amino acids or three contiguous amino acids. The deletion segments may be non-contiguous, or two or more or all of the deletion segments may be contiguous.

In particular embodiments, the variant as intended herein displays at least 80%, at least 85%, preferably at least 90%, such as at least 91%, at least 92%, at least 93%, at least 94%, more preferably at least 95%, such as at least 96%, at least 97%, at least 98%, or at least 99% overall amino acid sequence identity to human kisspeptin or fragment thereof. In preferred embodiments, said variant displays at least 90% overall amino acid sequence identity to human kisspeptin or fragment thereof.

In particular embodiments, the kisspeptin receptor agonist comprises, consists essentially of or consists of a protein, polypeptide or peptide having at least 80%, at least 85%, preferably at least 90%, such as at least 91%, at least 92%, at least 93%, at least 94%, more preferably at least 95%, such as at least 96%, at least 97%, at least 98%, or at least 99% overall amino acid sequence identity, more preferably overall sequence identity, to human kisspeptin, such as set forth in SEQ ID NO: 20, 16, 17, 18 or 19.

In certain embodiments, the kisspeptin receptor agonist comprises, consists essentially of or consists of a protein, polypeptide or peptide having at most 20 (e.g. not more than 20, 19, 18, 17 or 16), at most 15 (e.g. not more than 15, 14, 13, 12 or 11), at most 10 (e.g. not more than 10, 9, 8, 7 or 6), or at most 5 (e.g. not more than 5, 4, 3, 2 or 1) chemically modified amino acid residues different from and/or in addition to the chemically modified amino acid residues present in human kisspeptin, such as set forth in SEQ ID NO: 20, 16, 17, 18 or 19.

In certain embodiments, the kisspeptin receptor agonist comprises, consists essentially of or consists of the amino acid sequence of human kisspeptin, such as set forth in SEQ ID NO: 20, 16, 17, 18 or 19, wherein at most about 70%, e.g., at most about 50% or at most about 40%, preferably at most 30%, e.g., at most about 20%, more preferably at most about 10%, and yet more preferably at most about 5% or even about 1% of the amino acid residues are chemically modified.

In certain embodiments, the kisspeptin receptor agonist comprises, consists essentially of or consists of a protein, polypeptide or peptide of which at most 20 (e.g. not more than 20, 19, 18, 17 or 16), at most 15 (e.g. not more than 15, 14, 13, 12 or 11), at most 10 (e.g. not more than 10, 9, 8, 7 or 6), or at most 5 (e.g. not more than 5, 4, 3, 2 or 1) of the natural amino acid residues of human kisspeptin, such as set forth in SEQ ID NO: 20, 16, 17, 18 or 19, are substituted by D-amino acid residues and/or non-natural amino acid residues.

In certain embodiments, the kisspeptin receptor agonist comprises, consists essentially of or consists of the amino acid sequence of human kisspeptin, such as set forth in SEQ ID NO: 20, 16, 17, 18 or 19, wherein at most about 70%, e.g., at most about 50% or at most about 40%, preferably at most 30%, e.g., at most about 20%, more preferably at most about 10%, and yet more preferably at most about 5% or even about 1% of the amino acid residues are substituted by D-amino acid residues and/or non-natural amino acid residues.

In particular embodiments, the kisspeptin receptor agonist comprises, consists essentially of or consists of a protein, polypeptide or peptide lacking at most 20 contiguous amino acid residues (e.g. not more than 20, 19, 18, 17, or 16 contiguous amino acid residues), preferably lacking at most 15 contiguous amino acid residues (e.g. not more than 15, 14, 13, 12, or 11 contiguous amino acid residues), more preferably lacking at most 10 contiguous amino acid residues (e.g. not more than 10, 9, 8, 7 or 6 contiguous amino acid residues), or even more preferably lacking at most 5 contiguous amino acid residues (e.g. not more than 5, 4, 3, 2 or 1 contiguous amino acid residue(s)) compared to human kisspeptin, such as set forth in SEQ ID NO: 20, 16, 17, 18 or 19.

In certain embodiments, the kisspeptin receptor agonist comprises, consists essentially of or consists of the amino acid sequence of human kisspeptin, such as set forth in SEQ ID NO: 20, 16, 17, 18 or 19, wherein at most about 70%, e.g., at most about 50% or at most about 40%, preferably at most 30%, e.g., at most about 20%, more preferably at most about 10%, and yet more preferably at most about 5% or even about 1% of the amino acid residues are deleted.

In particular embodiments, the kisspeptin receptor agonist comprises, consists essentially of or consists of a protein, polypeptide or peptide having at most 25, at most 20, at most 15, at most 10, or at most 5 (e.g. not more than 5, 4, 3, 2 or 1) single amino acid substitutions compared to human kisspeptin, such as set forth in SEQ ID NO: 20, 16, 17, 18 or 19.

In certain embodiments, the kisspeptin receptor agonist comprises, consists essentially of or consists of the amino acid sequence of human kisspeptin, such as set forth in SEQ ID NO: 20, 16, 17, 18 or 19, wherein at most about 20%, e.g., at most about 15% or at most about 10%, preferably at most 5%, e.g., at most about 3%, more preferably at most about 2%, and yet more preferably at most about 1% of the amino acid residues are substituted by single amino acid substitutions.

In particular embodiments, the kisspeptin receptor agonist comprises, consists essentially of or consists of a protein, polypeptide or peptide having at most 20, at most 15, at most 10, or at most 5 (e.g. not more than 5, 4, 3, 2 or 1) conservative amino acid substitutions compared to human kisspeptin, such as set forth in SEQ ID NO: 20, 16, 17, 18 or 19.

In certain embodiments, the kisspeptin receptor agonist comprises, consists essentially of or consists of the amino acid sequence of human kisspeptin, such as set forth in SEQ ID NO: 20, 16, 17, 18 or 19, wherein at most about 20%, e.g., at most about 15% or at most about 10%, preferably at most 5%, e.g., at most about 3%, more preferably at most about 2%, and yet more preferably at most about 1% of the amino acid residues are substituted by an amino acid for another with similar characteristics (i.e. conservative amino acid substitution).

Reference to “fragment or variant” or “variant or fragment” of any protein, polypeptide or peptide, also encompasses fragments of variants of such protein, polypeptide or peptide, and fragments or variants of such protein, polypeptide or peptide.

Particularly envisaged are biologically active fragments or variants of the recited proteins, polypeptides or peptides. The term “biologically active” is interchangeable with terms such as “functionally active” or “functional”, denoting that the fragment or variant at least partly retains the biological activity or intended functionality of the respective or corresponding protein, polypeptide or peptide. Reference to the “activity” of a protein, polypeptide or peptide may generally encompass any one or more aspects of the biological activity of the protein, polypeptide or peptide, such as without limitation any one or more aspects of its biochemical activity, enzymatic activity, signaling activity, interaction activity, ligand activity, and/or structural activity, e.g., within a cell, tissue, organ or an organism.

Preferably, a functionally active fragment or variant may retain at least about 20%, e.g., at least about 25%, or at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% of the intended biological activity or functionality compared with the corresponding protein, polypeptide or peptide. In certain embodiments, a functionally active fragment or variant may even display higher biological activity or functionality compared with the corresponding protein, polypeptide or peptide, for example may display at least about 100%, or at least about 150%, or at least about 200%, or at least about 300%, or at least about 400%, or at least about 500% of the intended biological activity or functionality compared with the corresponding protein, polypeptide or peptide. By means of an example, where the activity of a given protein, polypeptide or peptide can be readily measured in an assay with a quantitative output, for example an enzymatic assay or a signalling assay or a binding assay producing a quantifiable signal, a functionally active fragment or variant of the protein, polypeptide or peptide may produce a signal which is at least about 20%, or at least about 25%, or at least 30%, or at least about 40%, or at least about 50%, or at least 60%, more preferably at least about 70%, or at least 80%, or at least about 85%, or at least about 90%, or at least about 95%, or at least about 100%, or at least about 150%, or at least about 200%, or at least about 300%, or at least about 400%, or at least about 500% of the signal produced by the corresponding protein, polypeptide or peptide, such as by an equimolar amount of the corresponding protein, polypeptide or peptide.

By means of an example and not limitation, a biologically active fragment or variant of human kisspeptin will at least partly retain one or more aspects of the biological activity of the corresponding native or wild-type human kisspeptin, respectively. For example, reference to the biological activity of the kisspeptin may particularly denote the ability to bind to human kisspeptin receptor, or the ability to activate kisspeptin receptor-mediated signalling. In preferred embodiments, reference to the biological activity of the kisspeptin may particularly denote the ability to activate KISSR-mediated signalling, as discussed elsewhere in this specification.

In particular embodiments, the fragment comprises, consists essentially of or consists of the amino acid sequence YNWNSFGLRF (SEQ ID NO: 20). In further embodiments, the fragment comprises, the amino acid sequence YNWNSFGLRF (SEQ ID NO: 20) which is further elongated N-terminally and/or C-terminally, with one or more (such as one, two or three) contiguous amino acid residues which occur N-terminally or C-terminally of the amino acid sequence YNWNSFGLRF (SEQ ID NO: 20) in the amino acid sequence of native KISS1 proprotein, such as set forth in SEQ ID NO: 16. For example, the fragment may comprise, consist essentially of or consist of LPNYNWNSFGLRFGKR (SEQ ID NO: 21), LPNYNWNSFGLRF (SEQ ID NO: 19), YNWNSFGLRFGKR (SEQ ID NO: 22), PNYNWNSFGLRFGK (SEQ ID NO: 23), PNYNWNSFGLRF (SEQ ID NO: 24), YNWNSFGLRFGK (SEQ ID NO: 25), NYNWNSFGLRFG (SEQ ID NO: 26), NYNWNSFGLRF (SEQ ID NO: 27), YNWNSFGLRFG (SEQ ID NO: 28), YNWNSFGLRF (SEQ ID NO: 20), NWNSFGLRF (SEQ ID NO: 29) or YNWNSFGLR (SEQ ID NO: 30), preferably YNWNSFGLRF (SEQ ID NO: 20).

In particular embodiments, the fragment comprises, consists essentially of or consists of human kisspeptin-10, such as set forth in SEQ ID NO: 20. In other embodiments, the fragment comprises human kisspeptin-10, such as set forth in SEQ ID NO: 20, with added thereto directly N-terminally, one or more (such as one, two or three) contiguous amino acid residues which occur N-terminally of the amino acid sequence of human kisspeptin-10 in the amino acid sequence of native kisspeptin-54, such as set forth in SEQ ID NO: 17. If three of such amino acid residues are present N-terminally of the amino acid sequence of kisspeptin-10, the fragment will comprise, consist essentially of or consist of human kisspeptin-13. If four of such amino acid residues are present N-terminally of the amino acid sequence of kisspeptin-10, the fragment will comprise, consist essentially of or consist of human kisspeptin-14.

In particular embodiments, the fragment comprises, consists essentially of or consists of kisspeptin-13, such as set forth in SEQ ID NO: 19. In other embodiments, the fragment comprises kisspeptin-13, such as set forth in SEQ ID NO: 19, with added thereto directly N-terminally one or more (such as one, two or three) contiguous amino acid residues which occur N-terminally of the amino acid sequence of human kisspeptin-13 in the amino acid sequence of native kisspeptin-54, such as set forth in SEQ ID NO: 17.

In particular embodiments, the fragment comprises, consists essentially of or consists of kisspeptin-14, such as set forth in SEQ ID NO: 18. In other embodiments, the fragment comprises kisspeptin-14, such as set forth in SEQ ID NO: 18, with added thereto directly N-terminally one or more (such as one, two or three) contiguous amino acid residues which occur N-terminally of the amino acid sequence of human kisspeptin-14 in the amino acid sequence of native kisspeptin-54, such as set forth in SEQ ID NO: 17.

An agonist of the kisspeptin receptor as intended herein which is a protein, polypeptide, or peptide may be naturally occurring, for example may be isolated or purified from a naturally occurring source of the protein, polypeptide, or peptide (such as, without limitation, from a cultured human cell line expressing human kisspeptin), or may be produced by any recombinant, semi-synthetic or synthetic means, or combinations of such available in the art. For example, production of a protein, polypeptide, or peptide of interest may be achieved by introducing a recombinant nucleic acid encoding the protein, polypeptide, or peptide operably linked to suitable regulatory sequences into a suitable cell or organism, bringing said protein, polypeptide, or peptide to expression in said cell or organism, and isolating the expressed protein, polypeptide, or peptide from said cell or organism and/or from the surrounding culture medium or supernatant.

In particular embodiments, the N-terminus and/or the C-terminus of the agonist as intended herein, wherein the agonist is a protein, polypeptide, or peptide, is modified after synthesis. In more particular embodiments, the N-terminus of the agonist is acetylated and/or the C-terminus of the agonist is amidated.

In particular embodiments, the kisspeptin receptor agonist comprises, consists essentially of or consists of a peptidomimetic of kisspeptin as described herein or of a biologically active fragment or variant of kisspeptin as described herein, or a pharmaceutically acceptable salt thereof.

The skilled person will understand that if it is envisaged to express and secrete the agonist as intended herein by a host cell, the nucleic acid encoding the agonist as intended herein preferably encodes a precursor form of the agonist including an N-terminal signal peptide sequence. Alternatively, the nucleic acid encoding the agonist as intended herein may be comprised within a vector providing for a signal peptide. The signal peptide may be a homologous or heterologous signal peptide, depending on the host cell used for production of the agonist as intended herein. Furthermore, for prokaryotic expression of the agonist as intended herein, a protease cleavage site motif may be present C-terminally of said signal peptide and N-terminally of the agonist as intended herein.

In particular embodiments, the kisspeptin receptor agonist as intended herein or a nucleic acid encoding the agonist, and optionally a pharmaceutically acceptable carrier, is comprised in a pharmaceutical composition.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application, preferably intranasal mucosal application), parenteral administration (such as, e.g., subcutaneous (e.g., subcutaneous or intradermal injection or infusion), intravenous (I.V.), intramuscular, intraperitoneal or intrasternal injection or infusion), transdermal (e.g. using a transdermal patch) or transmucosal (such as, e.g., oral, sublingual, intranasal) administration, topical administration, rectal, vaginal or intra-tracheal instillation, and the like. Preferably, the route of administration is intranasal, more particularly intranasal mucosal, or transdermal or parenteral administration, such as in certain preferred embodiments intravenous or subcutaneous administration.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the agent as disclosed herein in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the agent as disclosed herein with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

In particular embodiments, the agonist of human kisspeptin receptor can be administered to the subject in the form of a pill, tablet, capsule, alcoholic or oily solution, syrup, emulsion or suspension.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agonists and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

When administered intranasally, illustrative examples of carriers include polyethylene glycol, phospholipids, glycols and glycolipids, sucrose, and/or methylcellulose, powder suspensions with or without bulking agents such as lactose and preservatives such as benzalkonium chloride, EDTA. In particular embodiments, the agonist of human kisspeptin receptor can be administered to the subject in the form of a nasal spray or nasal drops.

When administered transdermally, the agonist of human kisspeptin receptor can be administered to the subject in the form of a transdermal patch for application to the skin of the subject. Such a patch typically comprises a backing layer and a pharmaceutical composition-containing layer that is adapted to be in diffusional communication with the skin of the subject and to transmit therapeutically effective amounts of the pharmaceutical composition through the skin of the subject.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

In particular embodiments, the agonist as intended herein is the main or only active ingredient of the pharmaceutical composition.

In particular embodiments, the agonist may be administered to the subject by central nervous system-directed delivery systems, more preferably brain-directed delivery systems. Non-limiting examples of brain-directed delivery systems include blood-brain barrier shuttle peptides (also known as molecular peptide vectors), anti-transferrin receptor antibodies, cell-penetrating peptides, and chitosan amphiphile nanoparticles.

In particular embodiments, the agonist may be administered to the subject using blood-brain barrier shuttle peptides. A number of shuttle peptides are known to be capable of mediating transfer of peptides across the BBB. Non-limiting examples of BBB shuttles include Angiopep-2, ApoB (3371-3409), ApoE (159-167), Peptide-22, THR, CRT, Leptin30, RVG29, CDX, Apamin, MiniAp-4, glutathione (GSH), G23, g7, TGN, TAT(47-57), SynB1, Diketopiperazines, and PhPro. When administering the agonist using BBB shuttle peptides, the agonist may either be directly conjugated to a BBB shuttle peptide or the agonist may be incorporated into a nanocarrier (e.g. liposome) which is coated with BBB shuttle peptides (e.g. G-Technology®). In particular embodiments, the agonist may be administered to the subject by conjugating the agonist to shuttle peptide Angiopep-2 or GSH.

Given the desired effect upon the administration of the agonist as intended herein, including treating a disorder of sexual desire in human females, enhancing libido in human females and inducing sexual arousal, the agonist is preferably administered to the subject within a reasonably short time prior to sexual activity.

In particular embodiments, the agonist is administered prior to sexual activity. When the agonist is administered prior to sexual activity, the sexual activity is typically foreseen and/or desired, meaning that the subject to which the agonist is administered typically knows prior to being administered the agonist when the sexual activity is about to take place. In more particular embodiments, the agonist is administered at most 5 hours, at most 4 hours, at most 3 hours, at most 2 hours, at most 1 hour, at most 0.5 hour, or at most 0.25 hour prior to sexual activity. In more particular embodiments, the agonist is administered from 4 hours to 0.25 hour, from 3 hours to 0.5, from 2 hours to 0.5 hour or from 1 hour to 0.5 hour prior to sexual activity.

Without limitation, depending on the type and severity of the disease, the agonist may be administered once prior to sexual activity. In particular embodiments, there is at least one day (e.g. one, two, three or four), preferably at least two days, between two consecutive administrations of the agonist. In particular embodiments, the agonist is not administered on two consecutive days or is not administered for a prolonged period of time.

Accordingly, in particular embodiments, the agonist is administered intranasally, transdermally, orally, intravenously or subcutaneously.

Administration can be by periodic injections of a bolus of the agonist or pharmaceutical composition comprising the agonist or can be uninterrupted or continuous by intravenous administration from a reservoir which is external (e.g., an IV bag) or internal (e.g., implantable pump). Accordingly, in particular embodiments, the agonist is administered by intravenous bolus injection, subcutaneous bolus injection, or intravenous infusion, preferably by subcutaneous bolus injection.

In particular embodiments, the agonist is administered by intravenous bolus injection, wherein the volume of the bolus is from 0.5 to 1 ml.

In particular embodiments, the agonist is administered by intravenous bolus injection, wherein the volume of the bolus is from 0.5 to 1 ml, preferably 0.5 ml. In particular embodiments, the concentration of the agonist when administered by intravenous bolus injection is from 0.1 to 10 nmol per kg body weight.

In particular embodiments, the agonist is administered by intravenous infusion, wherein the infusion rate is from 0.5 to 2, from 0.75 to 1.5, or from 1 to 1.25 nmol per kilogram bodyweight per hour, preferably from 0.5 to 1.25 nmol per kilogram bodyweight per hour. For example, the infusion rate may be about 1 nmol per kilogram bodyweight per hour.

In particular embodiments, the agonist is administered by intravenous infusion, wherein the infusion duration is from 10 minutes to 2 hours, from 30 minutes to 2 hours, from 30 minutes to 1.5 hours, from 45 minutes to 1.5 hours, or from 45 minutes to 1.25 hours. Preferably, the infusion duration is from 15 minutes to 1.5 hour.

The methods and uses as taught herein allow administering a therapeutically and/or prophylactically effective amount of an agonist as intended herein in subjects having a disorder of sexual desire which will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated. The term “prophylactically effective amount” refers to an amount of an active compound or pharmaceutical agent that inhibits or delays in a subject the onset of a disorder as being sought by a researcher, veterinarian, medical doctor or other clinician. Methods are known in the art for determining therapeutically and/or prophylactically effective doses of an agonist as intended herein.

The term “therapeutically effective dose” as used herein refers to an amount of an agonist as intended herein, that when administered brings about a positive therapeutic response with respect to treatment of a patient having a disorder of sexual desire.

Appropriate therapeutically effective doses of an agonist as intended herein, may be determined by a qualified physician with due regard to the nature of the disease condition and severity, and the age, size and condition of the patient.

Toxicity and therapeutic efficacy of the agonist as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

The dosage or amount of the agonist as intended herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the subject to be treated, efficacy, metabolic stability and duration of action of the agonist or pharmaceutical composition comprising the agonist used, on whether the therapy is acute or chronic or prophylactic, or on whether other active compounds are administered in addition to the agonist of the invention. In order to optimize therapeutic efficacy, the agonist as intended herein can be first administered at different dosing regimens. Typically, levels of the agonist in a tissue can be monitored using appropriate screening assays as part of a clinical testing procedure, e.g., to determine the efficacy of a given treatment regimen. The frequency and timing of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

In particular embodiments, the agonist is administered at a molar amount from 0.1 to 10, from 0.1 to 9, from 0.1 to 8, from 0.1 to 7, from 0.15 to 9, from 0.2 to 8, from 0.25 to 7.5, from 0.3 to 7, from 0.35 to 6.5, from 0.4 to 6, or from 0.5 to 5 nmol per kg body weight. Preferably, the agonist is administered at a molar amount from 0.1 to 10 nmol per kg body weight, such as at a molar amount of about 6 nmol per kg body weight. By means of example and without limitation, the agonist as intended herein may be administered at about 5.8, about 5.9, about 6.0, about 6.1, about 6.2, about 6.3, about 6.4, about 6.5, about 6.6, about 6.7, about 6.8, about 6.9 or about 7.0 nmol per kg body weight.

The terms “treat” or “treatment” encompass both the therapeutic treatment of an already developed disease or condition, as well as prophylactic or preventive measures, wherein the aim is to prevent or lessen the chances of incidence of an undesired affliction, such as to prevent occurrence, development and progression of a disorder of sexual desire. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like.

Except when noted, the terms “subject” or “patient” can be used interchangeably and refer to animals, preferably warm-blooded animals, more preferably vertebrates, even more preferably mammals, still more preferably primates, and specifically includes human patients and non-human mammals and primates. Preferred subjects are human subjects. The term “patient” is particularly used to refer to subjects in need of therapeutic treatment, more particularly subjects that would benefit from treatment of a given condition, particularly a disorder of sexual desire. Such subjects may include, without limitation, those that have been diagnosed with said condition, those prone to develop said condition and/or those in who said condition is to be prevented. On the other hand, the term “subject” is particularly used to refer to subjects who are not diagnosed with a given condition, particularly a disorder of sexual desire, but who would nevertheless benefit from the administration of the agonist in a non-therapeutic manner.

In particular embodiments, the human female, the human female patient or the human female subject is an adult human female. The term “adult” refers to a fully developed and (sexual) mature subject. The threshold of adulthood is typically associated with social and legal concepts. Preferably, the minimal age of the subject entering adulthood is at least 16 years old, at least 17 years old, at least 18 years old, at least 19 years old, at least 20 years old or at least 21 years old, preferably at least 16 years old.

In particular embodiments, the human female, the human female patient or the human female subject is a pre-menopausal, menopausal or post-menopausal adult female.

In particular embodiments, the human female, the human female patient or the human female subject is a human female undergoing hormone therapy or not undergoing hormone therapy. Non-limiting examples of hormone therapy include hormone replacement therapy (HRT) and hormonal contraception.

The two major biologically active estrogens in non-pregnant humans are estrone (E1) and estradiol (E2). A third bioactive estrogen, estriol (E3), is the main pregnancy estrogen, but plays no significant role in non-pregnant women. Postmenopausal women typically have a lower serum concentration of both estrone and estradiol.

In particular embodiments, the human female, the human female patient or the human female subject has a serum concentration of estrone (E1) from 17 to 200 pg/ml and a serum concentration of estradiol (E2) from 15 to 350 pg/ml.

In particular embodiments, the human female has a serum concentration of estrone (E1) from 7 to 40 pg/ml and a serum concentration of estradiol (E2) of less than 10 pg/ml.

In particular embodiments, the human female, the human female patient or the human female subject is aged less than 45 years, aged from 45 to 55 years or aged above 55 years.

The term “disorder of sexual desire”, “Sexual Interest Disorder (SID)” or “Sexual Desire Disorder (SDD)” as used herein, refers to any disease or disorder characterized by the general or situational lack or absence of sexual desire or libido for sexual activity and/or of sexual fantasies for a prolonged period of time, such as for minimum 6 months. Non-limiting examples of disorders of sexual desire include sexual aversion disorder (SAD) and hypoactive sexual desire disorder (HSDD).

In particular embodiments, the disorder of sexual desire is selected from the group consisting of SAD and HSDD. In preferred embodiments, the disorder of sexual desire is HSDD. The term “sexual aversion disorder” or “SAD” as used herein refers to a medical condition defined by persistent or recurrent extreme aversion to, and avoidance of, all or substantially all, genital sexual contact with a sexual partner.

The term “hypoactive sexual desire disorder” or “HSDD” as used herein refers to a persistent lack or absence of sexual desire for sexual activity and/or of sexual fantasies for a prolonged period of time that causes marked distress or interpersonal difficulty, as judged by a clinician. Typical symptoms associated with HSDD include distress, reduced or no initiation of sexual activity and absent or reduced interest in sexual activity, sexual thoughts or fantasies, sexual excitement or pleasure during most sexual activity, sexual interest or arousal in response to internal or external cues, genital or nonessential sensations during sexual activity, but are not limited thereto.

In particular embodiments, HSDD comprises absence of sexual desire, loss of sexual desire, or decrease in sexual desire. In particular embodiments, HSDD is general or situational HSDD. General HSDD typically refers to a lack of sexual desire which occurs in all situations, while on the other hand, in situational HSDD, the subject may still have sexual desire, but lacks sexual desire upon certain situations, for example, for its current partner. In particular embodiments, HSDD is acquired or lifelong.

In particular embodiments, the agonist is administered in combination with one or more other therapeutic suitable for treating the disorder of sexual desire in the human female. Such therapeutic can be a male pheromone or a compound with pheromone-like properties, such as androstadienone (also known as androsta-4,16-dien-3-one). More particularly, neuroimaging studies have shown that exposure to androstadienone increased the activity of the hypothalamus in heterosexual woman, but not, or to a lesser extend in heterosexual men.

In particular embodiments, the one or more other therapeutic is selected from the group consisting of androstadienone, flibanserin, testosterone, prasterone, trazodone, bremelanotide, bupropion, buspirone, sildenafil, lasofoxifene, BP-101, PL-6983, TGFK09SD, and combinations thereof.

In particular embodiments, the one or more other therapeutic is selected from the group consisting of the combination of bupropion and trazodone, the combination of buspirone and testosterone, and the combination of sildenafil and testosterone.

The agonist as described herein may also be used to enhance a temporary (e.g. less than 6 months) decrease in the libido of a human female subject or to further enhance the libido in a human female subject. A temporary decrease in libido is typically considered to fall within the ranges of a normal, healthy, libido, if the decrease in libido is not considered to be a disorder or disease, as judged by a medical doctor or other clinician.

Accordingly, a further aspect provides a non-therapeutic method of enhancing or maintaining libido in a human female subject, comprising administering to said subject an amount of an agonist of human kisspeptin receptor sufficient to enhance libido in said subject. The term “non-therapeutic” as used herein, refers to the intention to increase the level of health and/or well-being of an otherwise healthy individual or alternatively, of an individual who is apparently not suffering from (e.g., not diagnosed with) any disorders or diseases relating to the intended method or use.

In particular embodiments, the libido in the human female subject is at least 1.1-fold more, at least 1.2-fold more, at least 1.3-fold more, at least 1.4-fold more, at least 1.5-fold more, at least 2-fold more, at least 3-fold more, at least 4-fold more, or at least 5-fold more compared to the libido in the human female subject prior to the administration of the agonist as intended herein or induced by a neutral substance or negative control, for example as reported by the human female subject, or as reported by the sexual partner of the human female subject, or as determined by vaginal blood flow upon exposal to erotic movies or pictures as described in Woodard and Diamond, Physiologic measures of sexual function in women: a review. Fertil. Steril., 2009. 92(1):19-34.

In line therewith, a further aspect provides a non-therapeutic method of inducing sexual arousal in a human female subject, comprising administering to said subject an amount of an agonist of human kisspeptin receptor sufficient to induce sexual arousal in said subject. In particular embodiments, the non-therapeutic methods as disclosed herein comprise administering to said subject an amount of an agonist of human kisspeptin receptor sufficient to enhance libido or to induce sexual arousal, respectively, by a transdermal patch, a nasal spray, intravenous bolus injection, subcutaneous bolus injection, or intravenous infusion.

Similarly, a further aspect provides the non-therapeutic use of an agonist of human kisspeptin receptor for enhancing libido or for inducing sexual arousal in a human female subject, wherein said agonist is administered to said subject in an amount sufficient to enhance libido in said subject.

The skilled person will understand that the particular embodiments with regard to the agonist, the pharmaceutical compositions, the dosages, and the means of administration as described elsewhere in this specification for the use in the treatment or the method of treatment, also apply for the methods and uses for enhancing libido or for inducing sexual arousal in a human female subject, as taught herein.

The present application also provides aspects and embodiments as set forth in the following Statements:

Statement 1. An agonist of human kisspeptin receptor (e.g. KISS1R) for use in a method of treating a disorder of sexual desire in human females.

Statement 2. The agonist for use according to statement 1, wherein the agonist is human kisspeptin or a biologically active fragment or variant of human kisspeptin, or a pharmaceutically acceptable salt thereof.

Statement 3. The agonist for use according to statement 2, wherein said fragment comprises or consists of the amino acid sequence YNWNSFGLRF (SEQ ID NO: 20).

Statement 4. The agonist for use according to statement 2 or 3, wherein said variant displays at least 90% overall amino acid sequence identity to wild-type human kisspeptin or fragment thereof.

Statement 5. The agonist for use according to any one of statements 2 to 4, wherein said variant comprises one or more non-naturally occurring amino acids, chemically modified amino acids and/or D-amino acids.

Statement 6. The agonist for use according to any one of statements 1 to 5, wherein the agonist is selected from the group consisting of human kisspeptin-54, human kisspeptin-14, human kisspeptin-13, human kisspeptin-10, pharmaceutically acceptable salts thereof, and combinations thereof.

Statement 7. The agonist for use according to any one of statements 1 to 6, wherein the agonist is human kisspeptin-10 or a pharmaceutically acceptable salt thereof.

Statement 8. A method of treating a disorder of sexual desire in a human female patient, comprising administering to said patient a therapeutically effective amount of an agonist of human kisspeptin receptor.

Statement 9. The method according to statement 8, wherein the agonist is as defined in any one of statements 1 to 7.

Statement 10. The agonist for use according to any one of statements 1 to 7, or the method according to statement 8 or 9, wherein the disorder of sexual desire is hypoactive sexual desire disorder (HSDD).

Statement 11. The agonist for use according to statement 10, or the method according to statement 10, wherein HSDD comprises absence of sexual desire, loss of sexual desire, or decrease in sexual desire.

Statement 12. A non-therapeutic method of enhancing libido in a human female subject, comprising administering to said subject an amount of an agonist of human kisspeptin receptor sufficient to enhance libido in said subject.

Statement 13. A non-therapeutic method of inducing sexual arousal in a human female subject, comprising administering to said subject an amount of an agonist of human kisspeptin receptor sufficient to induce sexual arousal in said subject.

Statement 14. The method of statement 12 or 13, wherein the agonist is as defined in any one of statements 1 to 7.

Statement 15. The agonist for use according to any one of statements 1 to 7, 10 or 11, or the method according to any one of statements 8 to 14, wherein the agonist is administered prior to sexual activity.

Statement 16. The agonist for use according to any one of statements 1 to 7, 10, 11 or 15, or the method according to any one of statements 8 to 15, wherein the agonist is administered at a molar amount between 0.1 and 10 nmol per kg body weight.

Statement 17. The agonist for use according to any one of statements 1 to 7, 10, 11, 15 or 16, or the method according to any one of statements 8 to 16, wherein the agonist is administered intranasally, orally, transdermally, intravenously or subcutaneously.

Statement 18. The agonist for use according to statement 17, or the method according to statement 17, wherein the agonist is administered by a transdermal patch, a nasal spray, intravenous bolus injection, subcutaneous bolus injection, or intravenous infusion.

Statement 19. The agonist for use according to any one of statements 1 to 7, 10, 11, or 15 to 18, or the method according to any one of statements 8 to 18, wherein the agonist is administered in combination with one or more other therapeutic suitable for treating the disorder of sexual desire in the human female.

Statement 20. The agonist for use according to statement 19, or the method according to statement 19, wherein the one or more other therapeutic is selected from the group consisting of androstadienone, flibanserin, testosterone, prasterone, trazodone, bremelanotide, bupropion, buspirone, sildenafil, lasofoxifene, BP-101, PL-6983, TGFK09SD, and combinations thereof.

Statement 21. The agonist for use according to statement 19, or the method according to statement 19, wherein the one or more other therapeutic is selected from the group consisting of the combination of bupropion and trazodone, the combination of buspirone and testosterone, and the combination of sildenafil and testosterone.

Statement 22. Human kisspeptin or a biologically active fragment or variant of human kisspeptin, or a pharmaceutically acceptable salt thereof for use in a method of treating a disorder of sexual desire in human females.

Statement 23. Human kisspeptin or the biologically active fragment or variant thereof for use according to statement 22, wherein said fragment comprises or consists of the amino acid sequence YNWNSFGLRF (SEQ ID NO: 20).

Statement 24. Human kisspeptin or the biologically active fragment or variant thereof for use according to statement 22 or 23, wherein said variant displays at least 90% overall amino acid sequence identity to wild-type human kisspeptin or fragment thereof.

Statement 25. Human kisspeptin or the biologically active fragment or variant thereof for use according to any of statements 22 to 24, wherein said variant comprises one or more non-naturally occurring amino acids, chemically modified amino acids and/or D-amino acids.

Statement 26. Human kisspeptin-54, human kisspeptin-14, human kisspeptin-13, human kisspeptin-10, pharmaceutically acceptable salt thereof, or a combination thereof for use in a method of treating a disorder of sexual desire in human females.

Statement 27. Human kisspeptin-10 or a pharmaceutically acceptable salt thereof for use in a method of treating a disorder of sexual desire in human females.

Statement 28. A method of treating a disorder of sexual desire in a human female patient, comprising administering to said patient a therapeutically effective amount of human kisspeptin or a biologically active fragment or variant of human kisspeptin, or a pharmaceutically acceptable salt thereof.

Statement 29. The method according to statement 28, wherein said fragment comprises or consists of the amino acid sequence YNWNSFGLRF (SEQ ID NO: 20).

Statement 30. The method according to statement 28 or 29, wherein said variant displays at least 90% overall amino acid sequence identity to wild-type human kisspeptin or fragment thereof.

Statement 31. The method according to any of statements 28 to 30, wherein said variant comprises one or more non-naturally occurring amino acids, chemically modified amino acids and/or D-amino acids.

Statement 32. A method of treating a disorder of sexual desire in a human female patient, comprising administering to said patient a therapeutically effective amount of human kisspeptin-54, human kisspeptin-14, human kisspeptin-13, human kisspeptin-10, pharmaceutically acceptable salt thereof, or a combination thereof.

Statement 33. A method of treating a disorder of sexual desire in a human female patient, comprising administering to said patient a therapeutically effective amount of human kisspeptin-10 or a pharmaceutically acceptable salt thereof.

Statement 34. Human kisspeptin or a biologically active fragment or variant of human kisspeptin, or a pharmaceutically acceptable salt thereof for use according to any one of statements 22 to 27, or the method according to any of statements 28 to 33, wherein the disorder of sexual desire is hypoactive sexual desire disorder (HSDD).

Statement 35. Human kisspeptin or a biologically active fragment or variant of human kisspeptin, or a pharmaceutically acceptable salt thereof for use according to statement 34, or the method according to statement 34, wherein HSDD comprises absence of sexual desire, loss of sexual desire, or decrease in sexual desire.

Statement 36. A non-therapeutic method of enhancing libido in a human female subject, comprising administering to said subject an amount of human kisspeptin or a biologically active fragment or variant of human kisspeptin, or a pharmaceutically acceptable salt thereof sufficient to enhance libido in said subject.

Statement 37. A non-therapeutic method of inducing sexual arousal in a human female subject, comprising administering to said subject an amount of human kisspeptin or a biologically active fragment or variant of human kisspeptin, or a pharmaceutically acceptable salt thereof sufficient to induce sexual arousal in said subject.

Statement 38. The method according to statement 36 or 37, wherein said fragment comprises or consists of the amino acid sequence YNWNSFGLRF (SEQ ID NO: 20).

Statement 39. The method according to any of statements 36 to 38, wherein said variant displays at least 90% overall amino acid sequence identity to wild-type human kisspeptin or fragment thereof.

Statement 40. The method according to any of statements 36 to 39, wherein said variant comprises one or more non-naturally occurring amino acids, chemically modified amino acids and/or D-amino acids.

Statement 41. A non-therapeutic method of enhancing libido in a human female subject, comprising administering to said subject an amount of human kisspeptin-54, human kisspeptin-14, human kisspeptin-13, human kisspeptin-10, pharmaceutically acceptable salt thereof, or a combination thereof sufficient to enhance libido in said subject.

Statement 42. A non-therapeutic method of inducing sexual arousal in a human female subject, comprising administering to said subject an amount of human kisspeptin-54, human kisspeptin-14, human kisspeptin-13, human kisspeptin-10, pharmaceutically acceptable salt thereof, or a combination thereof sufficient to induce sexual arousal in said subject.

Statement 43. A non-therapeutic method of enhancing libido in a human female subject, comprising administering to said subject an amount of human kisspeptin-10 or a pharmaceutically acceptable salt thereof sufficient to enhance libido in said subject.

Statement 44. A non-therapeutic method of inducing sexual arousal in a human female subject, comprising administering to said subject an amount of human kisspeptin-10 or a pharmaceutically acceptable salt thereof sufficient to induce sexual arousal in said subject.

Statement 45. Human kisspeptin or a biologically active fragment or variant of human kisspeptin, or a pharmaceutically acceptable salt thereof for use according to any one of statements 22-27, 34, 35 or the method according to any one of statements 28-33, 34, 35, or 36-44, wherein human kisspeptin or a biologically active fragment or variant of human kisspeptin, or a pharmaceutically acceptable salt thereof is administered prior to sexual activity.

Statement 46. Human kisspeptin or a biologically active fragment or variant of human kisspeptin, or a pharmaceutically acceptable salt thereof for use according to any one of statements 22-27, 34, 35, or 45 or the method according to any one of statements 28-33, 34, 35, or 36-45, wherein human kisspeptin or a biologically active fragment or variant of human kisspeptin, or a pharmaceutically acceptable salt thereof is administered at a molar amount between 0.1 and 10 nmol per kg body weight.

Statement 47. Human kisspeptin or a biologically active fragment or variant of human kisspeptin, or a pharmaceutically acceptable salt thereof for use according to any one of statements 22-27, 34, 35, 45 or 46 or the method according to any one of statements 28-33, 34, 35, or 36-46, wherein human kisspeptin or a biologically active fragment or variant of human kisspeptin, or a pharmaceutically acceptable salt thereof is administered intranasally, orally, transdermally, intravenously or subcutaneously, such as preferably by a transdermal patch, a nasal spray, intravenous bolus injection, subcutaneous bolus injection, or intravenous infusion.

Statement 48. Human kisspeptin or a biologically active fragment or variant of human kisspeptin, or a pharmaceutically acceptable salt thereof for use according to any one of statements 22-27, 34, 35, 45-47 or the method according to any one of statements 28-33, 34, 35, or 36-47, wherein human kisspeptin or a biologically active fragment or variant of human kisspeptin, or a pharmaceutically acceptable salt thereof is administered in combination with one or more other therapeutic suitable for treating the disorder of sexual desire in the human female, such as preferably wherein the one or more other therapeutic is selected from the group consisting of androstadienone, flibanserin, testosterone, prasterone, trazodone, bremelanotide, bupropion, buspirone, sildenafil, lasofoxifene, BP-101, PL-6983, TGFK09SD, and combinations thereof, such as preferably wherein the one or more other therapeutic is selected from the group consisting of the combination of bupropion and trazodone, the combination of buspirone and testosterone, and the combination of sildenafil and testosterone. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1. Female Sexual Behaviour in Mice is Controlled by Kisspeptin Neurons

Present inventors discovered and characterised a central regulatory hub orchestrating sexual behavior in female mice. More particularly, it was found that kisspeptin-synthesizing neurons in the rostral periventricular area of the third ventricle (RP3V) of the hypothalamus present a central hub in transferring olfactory information perceived through the vomeronasal pathway to the reproductive center of the neuroendocrine brain leading to sex specific mate preferences and mating behaviors.

### Material and Methods

### Mouse Models

Kisspeptin knockout (Kiss^{-/-})²², Kisspeptin-IRES-Cre (KissIC)²⁴, GnRH::Cre¹²; Dicer^{loxP/loxP 60}, R26-BIZ³¹ and nNOS kockout (nNOS−/−)^{54, 61} mouse strains have been previously described and validated, and are on different genetic backgrounds (see Table 1). All experiments were performed on adult (>8 weeks of age) female mice unless otherwise stated.

Animal care and experimental procedures were performed in accordance with the guidelines established by the institutional animal care and use committee of the Royal Netherlands Academy of Arts and Science and by the National Institutes of Health “Guide for the Care and Use of Research Animals, Eight Edition”, and were approved by the Ethical Committee for Animal Use of the Universities of Liege (Belgium), Saarland (Germany) and of Otago (New Zealand). Female mice were placed into individual cages under a reversed light/dark cycle (12 h:12 h light/dark; 21.00 h lights on and 9.00 lights off) with food and water ad libitum.

### Ovariectomy and Hormone Supplementation

Unless otherwise stated, females were ovariectomized in adulthood (>8 weeks of age) under general anesthesia after either subcutaneous (sc) injections of ketamine (80 mg kg⁻¹ per mouse) and medetomidine (Domitor, Pfizer, 1 mg kg⁻¹ per mouse) or under 5% isoflurane, in order to control for endogenous hormone concentrations and to prevent pregnancies upon repeated testing. At the same time, all females received a 5 mm-long Silastic capsule (inner diameter: 1.57 mm; outer diameter: 2.41 mm) containing crystalline 17β-estradiol (diluted 1:1 with cholesterol) subcutaneously in the neck. The dose of E₂ (E8875, Sigma) was based on a previous study⁶² showing that this treatment leads to estradiol levels similar to mice in estrus. At the end of surgery, females under ketamine/medetomidine anesthesia received a sc injection of atipamezole (Antisedan, Pfizer, 4 mg kg⁻¹ per mouse) to antagonize medetomidine-induced effects and accelerate recovery. In order to induce sexual receptivity at the day of testing, all females received a subcutaneous injection with progesterone (500 μg, P0130, Sigma) 3 h before the onset of the behavioral test, unless stated otherwise (for overview of all the different hormone treatments, see Table 1).

### Viruses and Stereotaxic Injections for Behavioral Testing

The AAV-flex-taCasp3-TEVp virus (abbreviated to “AAV-Casp3” (Vector Core, University of North Carolina)) was stereotaxically injected bilateral into the RP3V in KissIC mice (total: Cre⁻: n=14; Cre⁺: n=14). AAV-Casp3 uses the T2A peptide encoding sequence to ensure bicistronic expression of pro-taCasp3 and TEVp after Cre-mediated recombination. taCasp3 triggers cell-autonomous apoptosis, thereby minimizing toxicity to adjacent Cre⁻ cells²³.

AAV5-EF1a-DIO-hChR2(H134R)-mCherry-WPRE-pA virus (abbreviated to “AAV-ChR2” (Vector Core, University of North Carolina)) was stereotaxically (see below) injected bilaterally into the RP3V in KissIC mice (Cre⁻: n=4; Cre⁺: n=8) to selectively express the light-activated cation channel Channelrhodopsin-2 and mCherry⁵⁹ in kisspeptin neurons.

Mice were placed in a motorized stereotaxic frame (Neurostar, Germany) under 5% isoflurane anesthesia. The skull was exposed by a midline scalp incision, and the stereotaxic frame was aligned at Bregma using visual landmarks. After alignment of the head of the mice, a drill was placed above the skull at coordinates (according to the Paxinos Brain Atlas⁶³) corresponding to the anteroventral periventricular nuclei (RP3V; rostrocaudal, 0.2 mm; mediolateral, ±0.1 mm) and a hole drilled through the skull bone to expose the brain. A 33-gauge steel needle loaded with virus (AAV-Casp3 or AAV-ChR2) was slowly inserted through the hole until it penetrated to a depth of 5.8 mm. Virus (1 μl per brain site injected) was delivered at 100 nl min⁻¹ through a Hamilton syringe using a syringe pump (Harvard Apparatus). The needle was left in place for an additional 10 min to allow diffusion of the virus before being slowly removed. Following AAV-Casp3 injection, the hole was filled with dental cement and the skin was sutured. Following AAV-ChR2 injection, a bilateral cannula (200 μm core diameter; Doric Lenses) holding optical fibers with 45° oriented mirror tips was inserted into the RP3V at a distance of 0.25 mm (mediolateral) from the center of injection and further fixed to the skull with dental cement. Mice were allowed to recover on a heating pad and returned to their home cage after waking up. All mice received a sc injection with Caprofen (5 mg kg⁻¹) for post-operative analgesia.

### Viruses and Stereotaxic Injections for Electrophysiology

Adult female (>2 months old) heterozygous KissIC mice were group-housed under conditions of controlled temperature (22±2° C.) and lighting (12-hour light, 12-hour dark cycles) with ad libitum access to food and water. Mice were anesthetized, placed in a stereotaxic apparatus and given simultaneous bilateral 0.5 μL injections of AAV9-EF1-DIO-hChR2-(H134R)-mCherry-WPRE-hGH (2.2×10¹³ GC ml⁻¹; Penn Vector Core) into the RP3V (coordinates according to the Paxinos Brain Atlas⁶³), 0.2 mm anterior to Bregma and 5.8 mm in depth) at a rate of 100 nl min⁻¹. The syringes were left in situ for 3 min before and 10 min after the injections. Following a recovery period, mice were bilaterally ovariectomized under anesthesia and, after >2 weeks, received subcutaneous Silastic implants containing 17-β-estradiol (1 μg per 20 g body weight) according to Bronson⁶⁴. Implants were made of 17-β-estradiol dissolved in ethanol and mixed with medical grade adhesive (0.1 mg ml⁻¹ adhesive), which is then injected into 1 mm internal diameter Silastic tubing. Six days later, mice received a subcutaneous injection of estradiol benzoate (1 μg per 20 g body weight) in the morning and were used for electrophysiology the following day

### Cannula Implantation for ICV Kisspeptin Administration

Mice were placed in a motorized stereotaxic frame (Neurostar, Germany) under 5% isoflurane anesthesia. The skull was exposed by a midline scalp incision, and the stereotaxic frame was aligned at Bregma using visual landmarks. After alignment of the head of the mice, a drill was placed above the skull at coordinates corresponding to the lateral ventricle (lateral +1, anterior-posterior: −0.34; dorsoventral: −2.5) and a hole drilled through the skull bone to expose the brain. Then a 26-gauge cannula cut at 2 mm from pedestal was implanted and fixed to the skull with dental cement. A dummy was inserted to close the cannula until the behavioral experiment. Mice were allowed to recover on a heating pad and returned to their home cage after waking up. All mice received a sc injection with Temsegic (0.05 mg kg⁻¹) for post-operative analgesia.

### Removal of the VNO

One week after ovariectomy, subjects underwent either bilateral removal of the VNO or sham surgery (VNOx or VNOi groups)⁶⁵. Briefly, animals were placed on their back and the lower jaw was gently opened after general anesthesia. A midline incision was made in the soft palate extending rostrally from behind the first palatal ridge to the incisors, and the underlying bone was exposed by blunt dissection. In VNOi animals, the incision was then closed with reabsorbable sutures. For VNOx animals, the rostral end of the VNO was exposed by drilling, the caudal end of the vomer bone was cut, and the VNO was removed bilaterally with a gentle twisting motion. Bleeding was controlled using a blunted 18-gauge needle attached to a vacuum. Animals were carefully monitored after surgery for bleeding and or breathing difficulties.

### Ablation of the MOE

Two weeks after the removal of the VNO (VNOx females) or sham surgery (VNOi females), mice received an intranasal application of 10% ZnSO₄ to lesion the main olfactory epithelium (MOEx) or saline solution (MOEi) under general anesthesia⁶⁶.

### Kisspeptin Treatment

Kisspeptin-10 (Kp-10) was synthesized in Strasbourg, France (Sequence: Tyr-Asn-Trp-Asn-Ser-Phe-Gly-Leu-Arg-Tyr-NH2, NeoMPS; weight=25.7 mg). To determine whether kisspeptin stimulated female sexual behavior in wildtype C57BL/6J mice, females received a sc injection of Kp-10 at the dose of 0.52 μg kg⁻¹ (injection volume 100 μl) 2h before the lordosis test. Each animal was used as its own control, i.e. the animal was injected one day with Kp-10 and the other day with saline. Females were not injected with progesterone in this particular experiment. Injections were separated by at least 3 days.

When injected intracerebroventricularly, females were injected with 10.4 ng kg⁻¹ of Kp-10 (injection volume 2 μl), 1 h before the lordosis test through a cannula inserted into the lateral ventricle.

### GnRH Treatment

Two hours before mate preference test, female mice received a single sc injection of GnRH (0.025 mg kg⁻¹, Polypeptide Laboratories France SAS, SC087).

### SNAP Treatment

S-nitroso-N-acetyl-DL-Penicillamine (SNAP) (N398-Sigma) is a NO donor. In order to ascertain the most efficient activity of SNAP (8 mg kg⁻¹) was combined with the guanylate cyclase agonist BAY41-2272 (10 mg kg⁻¹; B8810—Sigma) one hour before injection. One hour before behavioral tests, female mice received a single subcutaneous injection (100 μl) of the cocktail SNAP+BAY 41-2272.

### Brain Slice Preparation for Electrophysiology

Mice were killed by cervical dislocation, decapitated and brains quickly removed. Coronal brain slices (200-250 μm) containing the rostral periventricular area of the third ventricle (RP3V) were cut with a vibratome (VT1000S; Leica) in an ice-cold solution containing (in mM): NaCl 87, KCl 2.5, NaHCO₃ 25, NaH₂PO₄ 1.25, CaCl₂ 0.5, MgCl₂ 6, glucose 25 and sucrose 75. Slices were then incubated at 30° C. for at least one hour in artificial cerebrospinal fluid (aCSF; in mM): NaCl 120, KCl 3, NaHCO₃ 26, NaH₂PO₄ 1, CaCl₂ 2.5, MgCl₂ 1.2 and glucose 10. All solutions were equilibrated with 95% O₂/5% CO₂.

### Cell-Attached Recordings and Light Stimulation

Slices were placed under an upright microscope fitted for epifluorescence (Olympus, Tokyo, Japan) and constantly perfused (1.5 ml min⁻¹) with warm (−30° C.) aCSF. mCherry-expressing RP3V neurons were first visualized by brief fluorescence illumination and subsequently approached using infrared differential interference contrast optics. Action potential firing was recorded in voltage clamp mode in the cell-attached loose patch configuration. Recording electrodes (3-5 MΩ) pulled from borosilicate capillaries (Warner Instruments, Hamden, Conn.) with a horizontal puller (Sutter Instruments, Navato, Calif.) were filled with aCSF including 10 mM HEPES. Low resistance seals (10-30 MΩ) were achieved by applying either no suction or the lowest amount of suction required to detect spikes. For ChR2 activation, blue light was delivered to the slice through a 40× immersion objective (0.8 NA, Olympus) via a 470 nm light emitting diode (LED, CoolLED) connected to the vertical illumination port of the microscope. Stimulation consisted of 1-15 s trains of blue light pulses (2 ms duration; approximately 0.25 mW) delivered at 10 Hz, repeated ten times every 60 s in each cell. Electrophysiological signals were recorded using a Multiclamp 700B amplifier (Molecular Devices, Sunnyvale, Calif.) connected to a Digidata 1440A digitizer (Molecular Devices). Signals were low-pass filtered at 3 kHz before being digitized at a rate of 10 kHz and stored on a personal computer. Signal acquisition and analysis was carried out with pClamp 10 (Molecular Devices). Spikes were detected using the threshold crossing method. In each cell, spike fidelity was calculated by dividing the number of light-evoked spikes by the number of blue light stimuli and expressed as a percentage.

### Behavioral Tests

All experimental females were brought into behavioral estrus by ovariectomy (OVX) in adulthood and combined treatment with estradiol (E) through a silastic capsule and an acute injection (3 h before testing) with progesterone (P), unless stated otherwise (for all details on hormone treatments, see Table 1). Females were always tested during the dark phase of the light/dark cycle. Finally, levels of female sexual behavior displayed by the control (wild-type) females vary as function of the background strain with 129SvJ females (Kiss^{+/+}) showing relatively low levels compared to C57BL6/j females.

### Assessment of the MOE Lesion

Anosmia was assessed by submitting females to the hidden cookie test⁶⁶. Briefly, female mice were food-deprived overnight. A small piece of a chocolate chip cookie was buried (approximately 1 cm deep) at a random location in a clean Plexiglas aquarium (35 cm long 25 cm high 19 cm wide) containing fresh sawdust. The time it took each mouse to find the cookie was recorded. The test lasted until the mouse had located the cookie or 10 min if the cookie was not found. All mice treated with ZnSO₄ failed to find the hidden cookie and were thus considered to be anosmic.

### Exposure to Odors in Bedding

Four groups of gonadally intact males (n=5 each) were placed in clean cages containing fresh sawdust. Bedding was collected 12h later and directly used as olfactory stimulus for the experimental females. Thirty-six hours before bedding exposure, all experimental females (singly housed) were placed on clean sawdust in 2 separate housing units to separate females which were going to be exposed to male bedding or to clean bedding as control (and thus to prevent the controls from being exposed to male odors). On the day of testing, females were injected with P (500 μg) to induce behavioral estrus. This hormonal treatment made the experimental females behaviorally receptive at the time of odor exposure. Three hours after P injection, 15 gr of fresh male-soiled or clean bedding was placed into the subject's own cage. Ninety minutes after bedding exposure, females were perfused with paraformaldehyde and brains were collected.

### Mate Preference Tests

To assess mate preferences shown in response to auditory and olfactory stimuli, we used a box (60 cm long×30 cm high×13 cm wide) that was divided into three compartments using perforated opaque partitions. The partitions contained perforated holes at a height of 8 cm to facilitate the diffusion of odors from the two side compartments to the middle compartment. Tests were performed during the dark phase of the light cycle (5 h after lights out). Animals were habituated to the three compartment box only once on the day before the behavioral experiments by placing them in the middle compartment for 10 min (with no stimulus animals placed in the two side compartments). On the day of testing, an intact male stimulus and an estrous female stimulus were placed in the lateral compartments with their own bedding to make the stimuli as odorous as possible. Three hours after receiving a progesterone injection (500 μg), the female subject was introduced into the middle compartment, and was observed for 10 min. The time the subject spent poking her nose through the holes of the partition or actively sniffing the bottom of the partition in front of the female versus male stimulus animal was recorded. A preference score was calculated by dividing the time spent investigating the male compartment minus the time spent investigating the female compartment by the total time spent investigating both compartments. A positive value of the preference score indicates a mate preference directed toward the stimulus male whereas a negative value indicates a mate preference directed toward the stimulus female (for details, see⁶⁷).

Lordosis Tests without Photostimulation

Females were subjected to weekly lordosis tests in a Plexiglas aquarium (37 cm long×17 cm high×21 cm wide). A sexually experienced male was placed alone in the aquarium and allowed to adapt for 15 min. Subsequently, 3 h after receiving a subcutaneous progesterone injection (500 μg, P0130, Sigma) to induce behavioral estrus, the lordosis responses of the female to the mounts of the stimulus male were recorded. The test lasted until the female received 10 mounts or 10 min had elapsed. For the first experiment (mating-induced Fos activation) ovary intact females were paired with males during 30 minutes. A lordosis quotient (LQ) was calculated by dividing the number of lordosis responses displayed by the female subjects by the number of mounts received (×100). Before each experimental condition (drug injection, cell ablation, optogenetic stimulation), all females were subjected to at least three lordosis tests (with progesterone) in order to acquire sufficient sexual experience and thus a significant LQ. Tests were performed during the dark phase of the light cycle (5 h after lights out; for details, see⁶⁷). For all details on the different hormone treatments (estradiol versus estradiol+progesterone), see Table 1.

Lordosis Tests with Photostimulation

Prior to the lordosis test, the cannula was connected to an optical fiber (Doric Lenses) which in turn was connected to a blue laser (wavelength=473 nm) via an optical rotatory join allowing free movements of the animal. The optic fiber was flexible and long enough to allow the female to freely behave and interact with the male. After 3 pretests, KissIC females (Cre⁻ and Cre⁺) were then divided in two groups. On test 4, half of the females received optogenetic stimulation (stimulated) whereas the other half did not (unstimulated). On test 5 (conducted one week later), unstimulated females received an optogenetic stimulation while previously stimulated females did not, thus each female acted as her own control. Blue light was delivered through the optic cable at 10 Hz as soon as the male approached the female (sniffing and showing mount attempt). The duration of the stimulation varied as a function of the male, i.e. the time it took him to mount the female, however this was never longer than 15 seconds. Tests were performed over 10 minutes and the number of mounts was recorded as well as the number of female lordosis responses. Importantly, in order to observe possible stimulatory effects of photostimulation on lordosis behavior, females were not injected with progesterone before the lordosis test, and were thus only on estradiol treatment (by Silastic capsule, previously described).

### Transcardial Perfusion and OCT Embedding for Histology

Female mice were anesthetized and perfused transcardially with saline followed immediately by 4% ice-cold paraformaldehyde. Brains were removed and postfixed in 4% paraformaldehyde for 2 hours. Brains were then cryoprotected in 30% sucrose⁶⁸ in PBS and when sunken, were embedded in Optimal Cutting Temperature compound (OCT, Tissue-Tek). A Glass box was placed in a slurry of ethanol and dry ice. The glass box was then partially filled with isopentane. The tissue was placed in a plastic cuvette filled with OCT, placed into the isopentane bath and rapidly frozen. Brains were subsequently stored at −80° C. prior to sectioning.

### Histological Assessment of VNO Removal

As previously described⁶⁵, snouts were removed immediately after perfusion, cleared of all soft tissue, and soaked for 30 min in rapid decalcifier (Apex Engineering Products). Decalcified snouts were then soaked overnight in 30% sucrose⁶⁸ at which time a 1:1 mixture of 30% sucrose and OCT was suctioned into the nasal passages. Snouts were then incubated for 4 h in the 1:1 solution and were finally frozen in OCT and stored at −80° C. Snouts were sectioned at 10 μm thickness on a cryostat. One section every 150 μm was transferred directly onto Superfrost Plus glass slides and dried overnight. Sections were rinsed and stained with Hemotoxylin and Eosin to assess the presence of blood clots in the nasal sinuses and to determine whether the VNO was completely removed. A total of 32 mice were used and upon examination, 8 were excluded because the VNO was not completely removed. No blood clots were detected in any of the animals.

Immunohistochemical Detection of c-Fos or Kisspeptin

Brain sections (30 μm thick) were cut on a Leica CM3050S cryostat. Forebrains were cut coronally from the rostral telencephalon to the posterior hypothalamus. Sections were saved in four different series, placed in antifreeze solution, and stored at −20° C.

Immunostaining was carried out on free-floating sections. All incubations were carried out at room temperature, and all washes of brain tissue sections were performed using Tris-buffered saline (TBS 0.05M) or Tris-buffered saline containing 0.1% Triton X-100 (TB ST). Briefly, sections were rinsed and endogenous peroxidase activity was quenched by incubating the sections for 30 min with 0.3% hydrogen peroxide. Non-specific binding sites were then blocked by incubating sections for 30 min with 5% normal goat serum (NGS) (Dako Cytomation, Denmark). Sections were then incubated either with a rabbit polyclonal antibody ( 1/5000 in TB ST-NGS 5%; anti-kisspeptin-10, AB9754, Chemicon, Millipore) raised against the decapeptide kisspeptin-10 (derived from the Kiss-1 gene product) for 48 h at 4° C. or with a rabbit polyclonal anti-c-Fos antibody ( 1/2000 in TBST-NGS 5%; c-Fos (4): sc-52R, Santa Cruz Inc.) raised against the N-terminus of c-Fos of human origin. Sections were then incubated for 1 h in avidin-biotin complex ( 1/800, ABC, Vector Laboratory, Burlingam, Calif.) and then reacted for 5 min with 3,3′ diaminobenzidine tetrahydrochloride (DAB Kit, Vector Laboratory). Sections were then washed, dried overnight, left in xylene (Sigma) for 15 min and coverslipped using Eukit (Fluka, Steinheim, Germany).

Immunohistochemical Detection of c-Fos and Kisspeptin

To determine the distribution of c-Fos and kisspeptin double-labeled neurons, ovary intact females in proestrus (activation of kisspeptin cells following mating) or ovariectomized females (VNOx/MOEx experiment) were perfused with 4% paraformaldehyde in 0.1 M PBS 90 min after the introduction of the male to the female (onset of behavioral testing) or 90 min after being placed in the empty testing arena for the unmated controls. Regarding male bedding exposure (VNOx/MOEx), females were perfused 90 min after the onset of odor exposure (male or clean bedding). For the dual immunohistochemistry, sections were first washed in 0.1 M PBS pH 7.4 (PBS), peroxidase activity was blocked in PBS solution with 0.3% H₂O₂, and then permeabilized in PBS-0.1% Triton-X100 (PBST) and saturated in 5% NGS in PBST. Immediately after this step, sections were incubated in diluted anti-c-Fos antibody overnight. On the following day, sections were washed in PBST and incubated in a goat anti-rabbit biotinylated secondary antibody (Dako, Prod. Ref. B0432, 0.75 μg ml⁻¹ PBST). Sections were then washed in PBST and incubated in the Vectastain Elite ABC Kit (Vector, Prod. Ref. PK6100). After development with the DAB Substrate Kit (Vector, SK-4100), in a black precipitate (3,3′-diaminobenzidine (DAB) plus Ni²⁺), sections were washed thoroughly in PBS, and residual peroxidase activity blocked in PBS solution with 0.3% H₂O₂. Sections were then permeabilized and blocked in 5% NGS-PBST and incubated in anti-kisspeptin-10 antibody for 72 h. Similar secondary antibody and ABC incubation steps were then performed. The developing reaction used in this step was a DAB brown precipitate, using the same kit. Following this, sections were mounted in Eukitt after being air-dried.

Immunohistochemical Detection of Barley Lectin and nNOS

In order to identify cells that are synaptically connected to kisspeptin neurons, KissIC/R26-BIZ mice were transcardially perfused with 4% paraformaldehyde at proestrus or metestrus/diestrus (determined via vaginal cytology). Brains were sectioned at 14|m and collected in series of five on SuperFrost Plus slides (Roth) and stored at −80° C. The transsynaptic tracer BL was immunohistologically detected using goat anti-wheat germ agglutinin (1:1000, Vector Laboratories) and has been described previously³¹. A Tyramide Signal Amplification Plus Biotin kit was used for signal amplification. Briefly, slides were washed 3× for 5 mins in PBS, incubated in ice-cold methanol with 0.3% H₂O₂ for 30 mins, washed 3× for 5 mins in TNT (0.1 M Tris, 0.15 M NaCl, 0.05% Tween 20), incubated for 10 mins in 0.5% Triton-X100 in PBS, washed 3× for 5 mins in TNT, blocked with TNB for 30 mins and incubated with anti-wheat germ agglutinin (1:1000) in TNB overnight at 4° C. in a humidified chamber. The next morning, slides were brought to RT for 2 hours, washed 3× for 5 mins in TNT and incubated with biotinylated anti-goat IgG (1:500) in TNB for one hour at RT. Slides were then washed 3× for 5 mins in TNT and incubated with streptavidin-conjugated horseradish peroxidase (1:100) in TNB for 30 mins at RT. Slides were then washed 3× for 5 mins in TNT and incubated for 10 mins in biotin plus amplification reagent (1:50) in 1× plus amplification diluent at RT. Slides were then washed 3× for 5 mins in TNT followed by incubation in streptavidin-conjugated Cy5 (1:500) in TNB for 30 mins at RT. Slides were then washed 3× for 5 minutes with TNT. The slides were incubated overnight at 4° C. followed by two hours at RT with rabbit anti-nNOS (1:300) in 0.1 M PBS containing 0.5% λ-carrageenan (Sigma) and 0.02% sodium azide. The sections were then treated with Cy3-conjugated donkey anti-rabbit (1:500) in PBS containing 0.5% λ-carrageenan (Sigma) and 0.02% sodium azide in PBS for one hour at RT. Nuclei were stained with bisbenzimide solution (1:2000 in 0.1 M PBS, 5 min at RT) and coverslipped with Fluoromount-G (Southern Biotech). Images were taken using either a Zeiss Axioskop or a Zeiss Axio Scan Z1 epifluorescence microscope.

Immunohistochemical Detection of mCherry

To immunohistochemically detect mCherry, slides were washed 3× for 5 mins in PBS, incubated in 0.3% Triton-X100, 5% donkey serum and 0.02% sodium azide in PBS for one hour at RT followed by incubation with anti-ds-red (1:1000, recognizes mCherry) in PBS containing 0.5% k-carrageenan (Sigma) and 0.02% sodium azide overnight at 4° C. The next day slides were washed 3× for 5 mins with PBS containing 0.5% tween 20 (PBS+tween), incubated with Cy3-conjugated donkey anti-rabbit in PBS containing 0.5% λ-carrageenan (Sigma) and 0.02% sodium azide and washed 3× for 5 minutes in PBS+tween. Nuclei were stained with bisbenzimide solution (1:2000 in 0.1 M PBS, 5 min at RT) and coverslipped with Fluoromount-G (Southern Biotech). Images were taken using either a Zeiss Axioskop or a Zeiss Axio Scan Z1 epifluorescence microscope.

### Quantification and Statistical Analysis

Kisspeptin-immunoreactive (-ir) cells bodies were counted manually and bilaterally in three to four adjacent brain sections (with an interval of 120 μm between them) delineating the RP3V (anteroventral periventricular area+Periventricular preoptic zone) using a Zeiss Axioskop microscope (40× objective). Cell counts are expressed as mean number per section for each experimental condition. Analysis of kisspeptin-ir density in the ARC was performed as previously described⁶⁹. To analyze kisspeptin/c-Fos double labeling, three to four sections were selected from the RP3V and the total number of kisspeptin and kisspeptin/c-Fos co-labeled cells were counted in order to obtain the percentage of kisspeptin cells expressing c-Fos immunoreactivity per section.

nNOS-ir and BL+nNOS-ir cell bodies were counted unilaterally in eight to ten sections containing the VMHv1 (Bregma −1.34 to −1.94 according to⁶³. Cell counts are expressed as mean number per section for each experimental condition. Statistical significance was determined using Bonferroni's multiple comparison test.

### Statistics

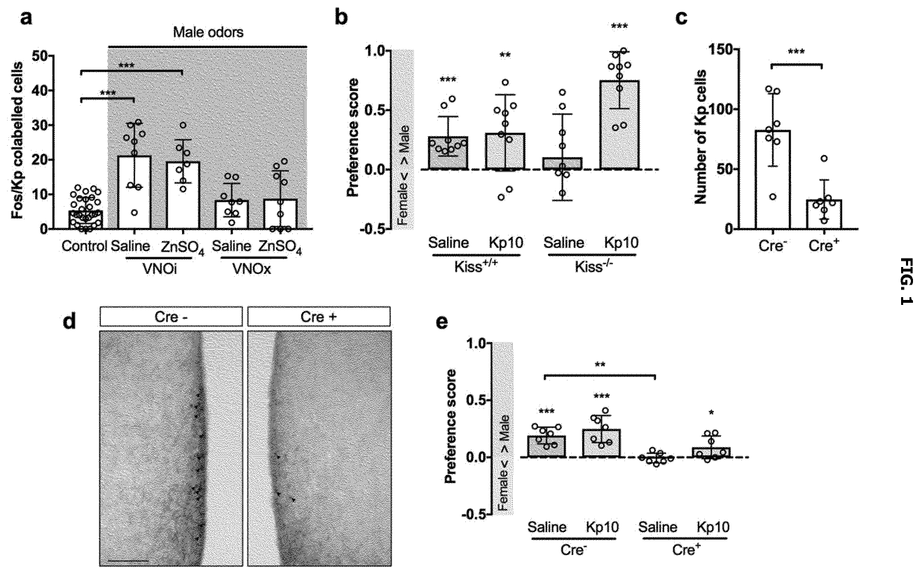

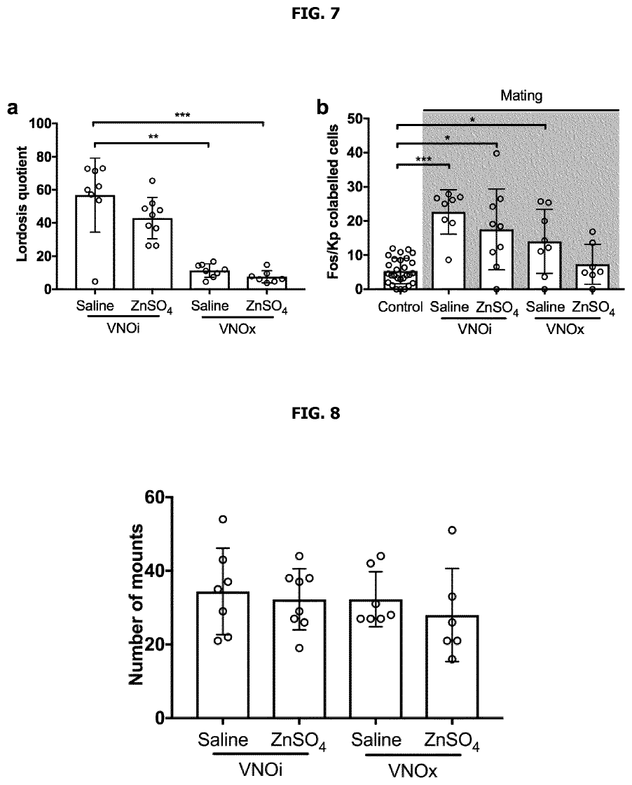

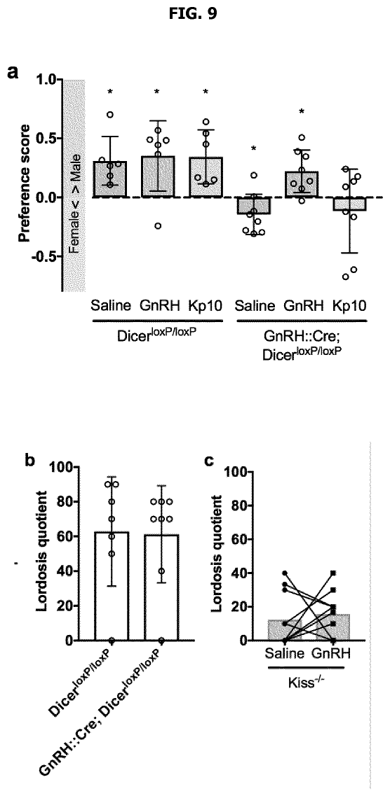

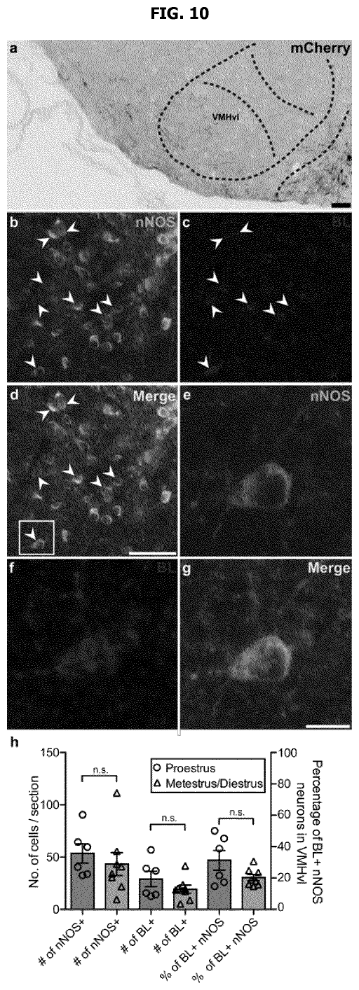

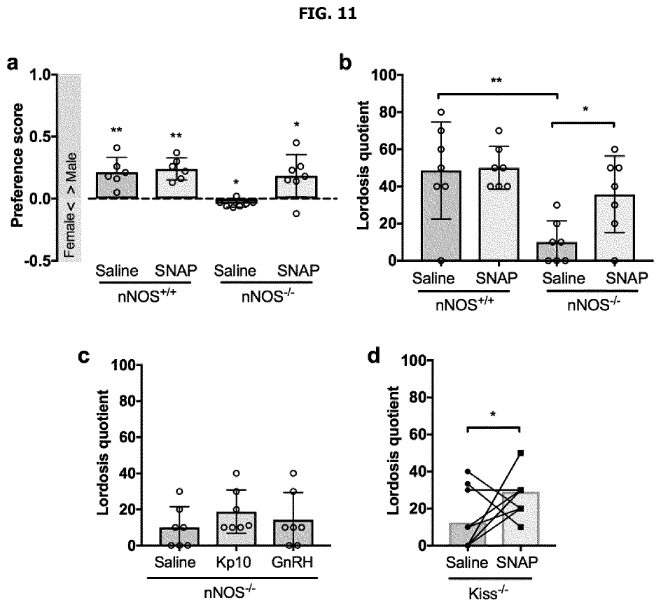

Randomization was not used in this study and no statistical methods were used to predetermine sample size. Investigators were blinded to the group allocation during experiments or data analysis. Data were analyzed using the GraphPad Prism 7 software. For all statistical comparisons, we first analyzed the data distribution with the Shapiro-Wilk test for normality. Preference score data were analyzed by comparison to hypothesized mean (H0: mean equal 0) using a non-directional One-sample two-tailed t test (FIGS. 1B, 1E, 9A, 11A)⁷⁰. For comparison of paired samples comparing two groups, statistical analysis was performed by using a paired-sample two-tailed t test (FIGS. 4B, 4D, 4F, 4I, 9C, 11D). Comparison of unpaired samples comparing two groups was then performed using an unpaired-sample two-tailed t test (FIGS. 1C, 4A, 4E). For comparison of more than two groups, an ANOVA test followed by a Tukey's (FIGS. 1E, 11B, 11C) or Bonferroni's (FIG. 10) two-tailed multiple comparisons test was used. Comparison of more than two data sets violating the normal distribution, the Kruskal-Wallis ANOVA two-tailed test followed by a Dunn's multiple comparison two-tailed test was used (FIGS. 1A, 7A, 7B). Comparison of two data sets violating the normal distribution, a one-sample Wilcoxon two-tailed test was used (FIGS. 4C, 9B).

TABLE 1
Genetic background of the mouse models used and hormonal treatments before the behavioural tests.

| | Mouse model | | | Treatments | | | | | | |
|---|---|---|---|---|---|---|---|---|---|---|
| | | Genetic | | | E₂ | Prog | | | Stimula- | |
| | Genotype | Background | Surgeries | Viruses* | (sc) | (sc) ** | Treatment | Injection | tion | N |
| | FIG. 1 | | | | | | | | | |
| a | WT | C57Bl/6J | OVX/VNOx | — | x | x | Saline/ZnSO₄ | intranasal | Clean or | 28/9/7/8/9 |
| | | | | | | | | | Male- | |
| | | | | | | | | | soiled | |
| | | | | | | | | | bedding | |
| b | Kiss^{+/+} & | 129/SvJ | OVX | — | x | x | Kp-10 | sc | Male and | 9 per |
| | Kiss^{-/-} | | | | | | | | female | genotype |
| | | | | | | | | | odors | |
| c-d | KissCre^{-/-} & | Mixed | OVX | AAV-flex- | x | x | — | — | Mating | 7 per |
| | KissCre^{+/+} | C57Bl/6J & | | taCasp3- | | | | | | genotype |
| | | 129/SvJ | | TEVp | | | | | | |
| e | KissCre^{-/-} & | Mixed | OVX | AAV-flex- | x | x | —/Kp10 | sc | Male and | 7 per |
| | KissCre^{+/+} | C57Bl/6J & | | taCasp3- | | | | | female | genotype |
| | | 129/SvJ | | TEVp | | | | | odors | |
| | FIG. 4 | | | | | | | | | |
| a | WT | C57Bl/6J | — | — | — | — | — | — | Mating | 3 (Unmated)/ |
| | | | | | | | | | | 5 (Mated) |
| b | WT | C57Bl/6J | OVX | — | x | — | Saline/ Kp10 | sc | Mating | 8 per group |
| c-d | Kiss^{+/+} & | 129/SvJ | OVX | — | x | x | −/Kp-10 | sc | Mating | 7 (Kiss+/+)/ |
| | Kiss^{-/-} | | | | | | | | | 10 (Kiss−/−) |
| e-f | KissCre^{-/-} & | Mixed | OVX | AAV-flex- | x | x | −/Kp10 | sc | Mating | 7 per |
| | KissCre^{+/+} | C57Bl/6J & | | taCasp3- | | | | | | genotype |
| | | 129/SvJ | | TEVp | | | | | | |
| I | KissCre^{-/-} & | Mixed | OVX | AAV5-EF1a- | x | − | Blue light*** | | Mating | 8 per |
| | KissCre^{+/+} | C57Bl/6J & | | DIO- | | | | | | genotype |
| | | 129/SvJ | | hChR2(H134R)- | | | | | | |
| | | | | mCherry- | | | | | | |
| | | | | WPRE-pA | | | | | | |
| | FIG.7 | | | | | | | | | |
| a | WT | C57Bl/6J | OVX/VNOx | — | x | x | Saline/ZnSO4 | intranasal | Mating | 8/9/8/7 |
| b | WT | C57Bl/6J | OVX/VNOx | — | x | x | Saline/ZnSO4 | intranasal | Mating | 28/8/9/8/7 |
| | FIG. 9 | | | | | | | | | |
| a | Dicer^{loxP/loxP} & | C57Bl/6J | OVX | — | x | x | Saline/Kp10 | sc | Male and | 6 |
| | GnRH::Cre; | | | | | | | | female | Dicer^{loxP/loxP} & |
| | Dicer^{loxP/loxP} | | | | | | | | odors | GnRH::Cre; |
| | | | | | | | | | | Dicer^{loxP/loxP} |
| a | Dicer^{loxP/loxP} & | C57Bl/6J | OVX | — | x | — | GnRH | sc | Male and | 6 |
| | GnRH::Cre; | | | | | | | | female | Dicer^{loxP/loxP} & |
| | Dicer^{loxP/loxP} | | | | | | | | odors | GnRH::Cre; |
| | | | | | | | | | | Dicer^{loxP/loxP} |
| b | Dicer^{loxP/loxP} & | C57Bl/6J | OVX | — | x | x | — | | Mating | 7/8 |
| | GnRH::Cre; | | | | | | | | | |
| | Dicer^{loxP/loxP} | | | | | | | | | |
| c | Kiss^{+/+} & | 129/SvJ | OVX | — | x | — | −/GnRH | sc | Mating | 10 per |
| | Kiss^{-/-} | | | | | | | | | treatment |
| | FIG. 10 | | | | | | | | | |
| a | KissIC/R26- | Mixed | — | AAV5-EF1a- | — | — | — | — | — | 3 (unknown |
| | BlZ | C57Bl/6J & | | DIO- | | | | | | estrous |
| | | 129/SvJ | | hChR2(H134R)- | | | | | | cycle stage) |
| | | | | mCherry- | | | | | | |
| | | | | WPRE-pA | | | | | | |
| b | KissIC/R26- | Mixed | — | — | — | — | — | — | — | 6 (proestrus) |
| | BlZ | C57Bl/6J & | | | | | | | | and 8 |
| | | 129/SvJ | | | | | | | | (metestrus/ |
| | | | | | | | | | | diestrus) |
| | FIG. 11 | | | | | | | | | |
| a | nNOS^{+/+} & | C57Bl/6J | OVX | — | x | x | Saline/SNAP | sc | Male and | 6 (nNOS^{+/+})/ |
| | nNOS^{-/-} | | | | | | | | female | 7 (nNOS^{-/-}) |
| | | | | | | | | | odors | |
| b | nNOS^{+/+} & | C57Bl/6J | OVX | — | x | x | Saline/SNAP | sc | Mating | 7 per group |
| | nNOS^{-/-} | | | | | | | | | |
| c | nNOS^{-/-} | C57Bl/6J | OVX | — | x | x | −/Kp10 | sc | Mating | 7 per group |
| c | nNOS^{-/-} | C57B1/6J | OVX | — | x | — | GnRH | sc | Mating | 7 per |
| | | | | | | | | | | treatment |
| d | Kiss^{-/-} | 129/SvJ | OVX | — | x | x | −/SNAP | sc | Mating | 10 per |
| | | | | | | | | | | treatment |
| | Supplementary FIGS. | | | | | | | | | |
| 1 | KissCre^{-/-} & | Mixed | OVX | AAV-flex- | x | — | — | — | — | 7 per |
| | KissCre^{+/+} | C57Bl/6J & | | taCasp3- | | | | | | genotype |
| | | 129/SvJ | | TEVp | | | | | | |
| 2-3 | WT | C57Bl/6J | — | — | — | — | — | — | Mating | 5 per group |
| 4 | WT | C57Bl/6J | OVX | — | x | x | Saline/Kp10 | icv | Mating | 5/7 |
| 5 | WT | C57Bl/6J | OVX | — | x | x | — | sc | Mating | 7/8/7/6 |

*Viruses were injected bilaterally into the RP3V.
**Progesterone was administered 3 h prior to behavioral experiments.
***Photostimulation at 10 Hz, 473 nm.
All experimental females were brought into behavioral estrus by adult ovariectomy and combined treatment with estradiol and progesterone unless stated otherwise. Furthermore, levels of female sexual behavior displayed by the control (wild-type) females vary as function of the background strain with 129/SvJ females showing relatively low levels compared to C57Bl/6J females.
Abbreviations:
WT, wild-type;
E2, estradiol;
P, progesterone;
sc, subcutaneous;
icv, intracerebroventricular;
Kp10, kisspeptin;
SNAP, S-nitroso-N-acetylpenicillamine;
RP3V, rostral periventricular area of the third ventricle of the hypothalamus

### Results

### Male Odors Activate RP3V Kisspeptin Neurons

Present inventors previously found that RP3V kisspeptin neurons are specifically activated by male odors derived from either urine¹⁹ or soiled bedding in female mice. To determine the olfactory input pathway impinging onto these cells, we selectively ablated the vomeronasal organ (VNO) by surgical removal and/or the main olfactory epithelium (MOE) by intranasal infusion with a zinc sulfate (ZnSO₄) solution in female C57Bl/6j mice (see general remarks on behavioral tests in Methods and Table 1 for details on hormonal treatments prior to testing). Next, male odor-triggered kisspeptin neuron activation was tested in these animals by using c-Fos as a marker. Removal of the VNO (VNOx), but not ablation of the MOE (MOEx), completely eliminated the ability of male odors contained in soiled bedding to activate RP3V kisspeptin neurons in ovariectomized female mice supplemented with estradiol and progesterone (OVX+E+P) (VNOi group vs Control group, P<0.001; MOEx group vs Control group, P=0.001; VNOx group vs Control group, P>0.99, all compared with the control group; Dunn's multiple comparison test; FIG. 1a). These results demonstrate that pheromonal input triggers c-Fos expression in kisspeptin neurons via the vomeronasal pathway. The number of kisspeptin neurons was not affected by either VNOx or zinc sulfate treatment.

### RP3V Kisspeptin Neurons Trigger Mate Preferences

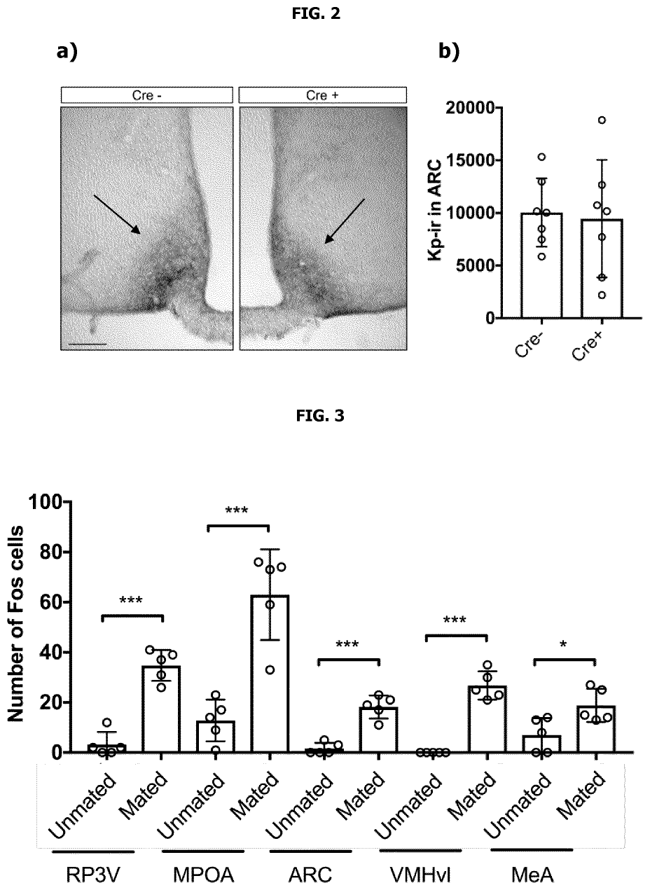

Since pheromonal cues are critical for mate recognition^{20, 21}, present inventors next asked whether the kisspeptin peptide plays a role in olfactory mate preference. To address this question, mice lacking a functional Kiss1 gene²² were analyzed. Kisspeptin knockout (Kiss^{-/-}; OVX+E+P) mice failed to show any male-directed preference (One-sample t test (H0: mean equals 0); P=0.42; FIG. 1b) whereas control littermates (OVX+E+P) displayed robust preferences for the male (One-sample t test (H0: mean equals 0); P<0.001; FIG. 1b). A single subcutaneous (sc) injection of kisspeptin (Kp-10; 0.52 μg kg⁻¹) triggered a male-directed preference in Kiss^{-/-} (OVX+E+P) females (One-sample t test (H0: mean equals 0); P<0.001; FIG. 1b). These data implicate the kisspeptin neuropeptide in olfactory-driven partner preference. Kisspeptin is, however, not only expressed in the RP3V but also in neurons located in the arcuate nucleus of the hypothalamus (ARC) in the adult rodent brain¹⁸. To analyze the specific role of the RP3V kisspeptin neuron population in olfactory mate preference, these cells were ablated by injecting an adeno-associated virus (AAV) encoding a Cre recombinase-dependent caspase 3²³ bilaterally into the RP3V of mice expressing Cre in kisspeptin neurons (KissIC)²⁶. Caspase 3 kills the Cre-expressing cells by inducing apoptosis²³. Stereotaxic viral delivery into the RP3V led to a 71% decrease in kisspeptin-immunoreactive cells in Cre⁺ females compared to control Cre⁻ animals (Unpaired t test; P<0.001; FIGS. 1c and 1d) suggesting efficient ablation of this neuronal population. In contrast, kisspeptin-immunoreactivity in the ARC was not affected in this experimental paradigm (Unpaired t test; P=0.81, FIG. 2). Strikingly, Cre⁺ females failed to show any male-directed preferences after acute ablation of the RP3V kisspeptin neuron population (One-sample t test (H0: mean equals 0); P=0.73; FIG. 1e), whereas a single sc injection of Kp-10 was sufficient to induce a male-directed preference in these female (OVX+E+P) mice (One-sample t test (H0: mean equals 0); P=0.041; FIG. 1e). Taken together, these data demonstrate that RP3V kisspeptin neurons could be an essential component of the neural circuits downstream of the vomeronasal organ mediating pheromone-driven mate preference in female mice.

### RP3V Kisspeptin Neurons are Essential for Lordosis

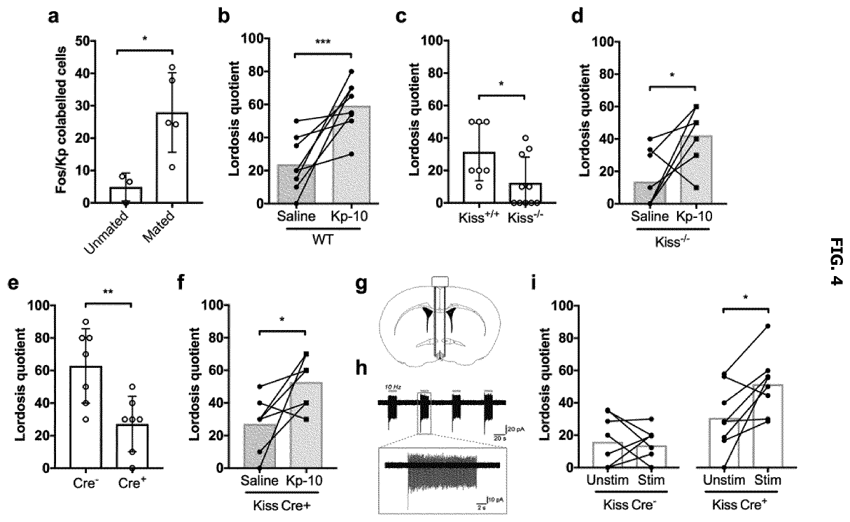

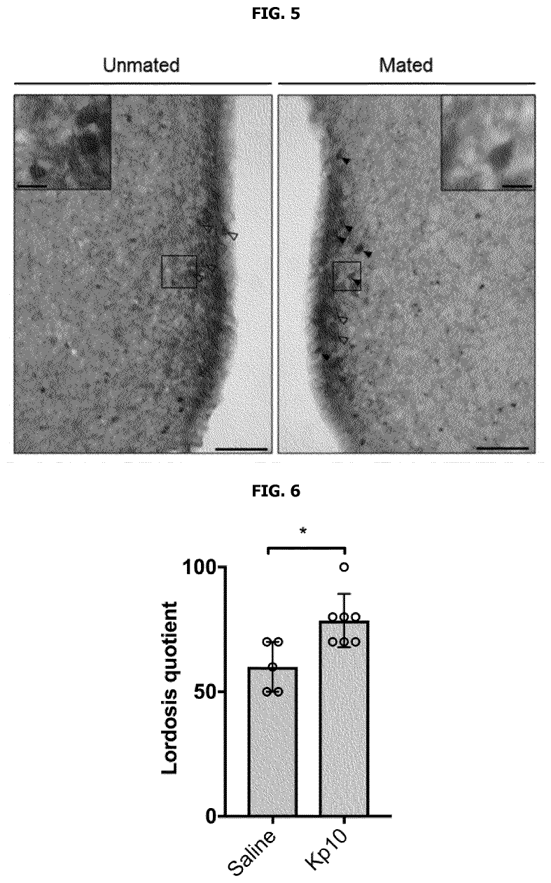

Pheromone-triggered mate preference ultimately leads to the display of copulatory behaviors. Present inventors therefore next investigated the role of the RP3V kisspeptin neurons in lordosis behavior, which is characterized by an arching of the back and an immobile posture by the female in response to male mounting. Consistent activation of hypothalamic neurons was found upon mating using c-Fos immunoreactivity as a marker (FIG. 3) in ovary intact, proestrous female mice. Specifically, ˜30% of RP3V kisspeptin neurons displayed c-Fos immunoreactivity in brain of intact females (Unpaired t test; P=0.022; FIG. 4a and FIG. 5) following this experimental paradigm. Present inventors next asked whether a single injection with Kp-10 is sufficient to stimulate lordosis behavior in female mice. It was found that a sc Kp-10 injection in OVX+E females robustly stimulated lordosis behavior (Paired t test; P<0.001; FIG. 4b). Likewise, an intracerebroventricular (icy) injection of Kp-10 (Paired t test; P=0.012; FIG. 6) also stimulated lordosis behavior in C57B16 (OVX+E+P) female mice (for details on hormone treatments, see Table 1). Next lordosis behavior in Kiss^{-/-} (OVX+E+P) females was analyzed and strong deficits (Mann-Whitney U test; P=0.029; FIG. 4c) were observed, which could be reversed by a single sc injection of Kp-10 (Paired t test; P<0.01; FIG. 4d). Consistent with this, acute ablation of ˜70% of kisspeptin neurons by bilateral injection of an AAV virus encoding a Cre recombinase-dependent caspase 3 into the RP3V of adult female (OVX+E+P) KissIC mice also led to profound deficits in lordosis behavior (Unpaired t test; P=0.006; FIG. 4e), which were reversible upon a single sc Kp-10 injection (Paired t test; P=0.03; FIG. 4f).

To test whether an activation of RP3V kisspeptin neurons is sufficient to trigger lordosis behavior, an AAV encoding a Cre-dependent channelrhodopsin (ChR2) was stereotaxically injected bilaterally into the RP3V of female KissIC mice (FIG. 4g). Blue light photostimulation (1-15 s at 10 Hz) elicited robust firing in virally transduced kisspeptin neurons with spike fidelity of 99% (FIG. 4h) in brain slice preparations. Photostimulation of RP3V kisspeptin neurons in vivo at 10 Hz for <15s per male mount also was successful in enhancing lordosis behavior in Cre⁺ (OVX+E) female mice (Paired t test; P=0.025; FIG. 4i) without enhancing lordosis expression in Cre⁻ (OVX+E) female mice injected with the AAV-ChR2 virus (Paired t test; P=0.72; FIG. 4i). Taken together, these experiments demonstrate that RP3V kisspeptin neurons are an integral part of the neural network involved in both mate preference and lordosis behavior in female mice.

### Olfactory Control of Lordosis Behavior

Next, the individual contribution of the vomeronasal pathway and the main olfactory system on RP3V kisspeptin neuron activation during sexual interaction with a male were determined. VNO removal (Dunn's multiple comparison test; P=0.006; FIG. 7a) but not the ablation of the MOE (Dunn's multiple comparison test following Kruskal Wallis test; P >0.99; FIG. 7a) disrupted lordosis behavior in C57B16/j (OVX+E+P) females (without affecting the male behavior towards the female; Dunn's multiple comparison test; VNOi/ZnSO4 vs VNOi/Saline P>0.99; VNOx/Saline vs VNOi/Saline P>0.99; VNOx/ZnSO4 vs VNOi/Saline P>0.99; FIG. 8) indicating that lordosis mainly depends on a functional vomeronasal pathway. While ablation of the VNO resulted in a dramatic attenuation in lordosis behavior, c-Fos expression in RP3V kisspeptin neurons remained significant (Dunn's multiple comparison test; P=0.01 compared to controls). This might reflect activation through the MOE by specific volatile odors only secreted by the male when in direct contact with the female, since ablation of both the VNO and the MOE in mice completely eliminated RP3V kisspeptin neuron activation (FIG. 7b). Regardless, activation of RP3V kisspeptin neurons exclusively through the main olfactory system when in direct contact with the male seems to be insufficient to trigger lordosis. Taken together, these data reveal that RP3V kisspeptin neurons are an essential part of a motivational neural pathway that is triggered by male olfactory cues detected and processed predominantly through the vomeronsal pathway, ultimately leading to the female adapting a specific mating posture facilitating intromission.

### Mate Preference, but not Lordosis, is GnRH-Dependent

RP3V kisspeptin neurons directly innervate GnRH neurons^{25, 26} and are implicated in generating the preovulatory LH surge^{16, 17, 24, 27, 28}. Kisspeptin can activate GnRH neurons via its canonical receptor Kiss1R, which is expressed in ˜95% of these cells^{14, 15, 29}. To test whether RP3V kisspeptin neurons act on GnRH neurons to drive olfactory mate preference and lordosis behavior, we used GnRH::Cre; Dicer^{loxP/loxP} females which are incapable of synthesizing and secreting GnRH in adulthood³⁰. GnRH::Cre; Dicer^{loxP/loxP} (OVX+E+P) females failed to show male-directed preferences and actually showed a preference for the female (One-sample t test (H0: mean equals 0); P=0.049; FIG. 9a). A single sc injection of GnRH (0.025 mg kg⁻¹) restored this behavior in these females supplemented with only estradiol (OVX+E) (One-sample t test (H0: mean equals 0); P=0.01; FIG. 9a) whereas a sc injection of Kp-10 failed to elicit a male-directed preference in female (OVX+E+P) GnRH::Cre; Dicer^{loxP/loxP} mice (One-sample t test (H0: mean equals 0); P=0.38; FIG. 9a). It was found that lordosis behavior was not affected in (OVX+E+P) GnRH::Cre; Dicer^{loxP/loxP} females (Mann-Whitney U test; P=0.79; FIG. 9b). These results suggest that although GnRH neurons are required for mate preference, they might not be essential for the expression of lordosis behavior. Consistent with this, GnRH injection into Kiss^{-/-} females failed to stimulate lordosis behavior in (OVX+E) mice (Paired t test test; P=0.65; FIG. 9c).

### Kisspeptin Control of Lordosis is Mediated by Nitric Oxide

To identify potential candidate neurons downstream of RP3V kisspeptin neurons other than GnRH neurons, ovary-intact female KissIC/R26-BIZ mice were used which express the transsynaptic tracer barley lectin (BL) exclusively in kisspeptin neurons³¹. Because BL is also expressed in ARC kisseptin neurons in these animals, a Cre-dependent mCherry adeno-associated virus bilaterally was injected into the RP3V to delineate the projections from RP3V kisspeptin neurons. A cluster of BL+ cells was observed in the ventrolateral part of the ventromedial hypothalamus (VMHv1), a brain area previously implicated in reproductive behaviors³². Subsequent immunohistochemical analyses (FIG. 10a-g) showed that a major subpopulation of the BL+neurons in the VMHv1 express neuronal nitric oxide synthase (nNOS), previously implicated in reproductive behaviors^{33, 34}. Furthermore, the tract tracing suggests that RP3V kisspeptin neurons project to the VMHv1 and therefore, these VMHv1 nNOS neurons may be downstream of RP3V kisspeptin neurons. Present inventors observed at proestrus that 31.27%±6.22% of nNOS neurons in the VMHv1 were BL+, whereas at diestrus 20.25%±1.94% contained the tracer, however this was not found to be statistically significant (P>0.05, Bonferroni Multiple Comparison test; FIG. 10h). Taken together, these data indicate that nNOS neurons within the VMHv1 are part of a neural pathway containing kisspeptin neurons.

To further dissect the functional role of NO signaling in this neural circuit important for sexual behavior, we next analyzed mate preference and lordosis behavior in mice deficient in nNOS. We found that nNOS knockout (nNOS^{-/-}) (OVX+E+P) females actually showed a small, albeit significant, preference for the female (One-sample t test (H0: mean equals 0); P=0.01; FIG. 11a). However, a male directed preference comparable to control littermates could be induced when injected sc with a cocktail of the nitric oxide donor SNAP and the guanylate cyclase agonist BAY 41-2272 (One-sample t test (H0: mean equals 0); P=0.03; FIG. 11a). Accordingly, nNOS^{-/-} (OVX+E+P) females also showed a strong decrease in lordosis behavior compared to control littermates (Tukey's multiple comparison test; P=0.002; FIG. 11b), which was restored by a sc injection of the cocktail SNAP+BAY 41-2272 (Tukey's multiple comparison test; P=0.04; FIG. 11b). By contrast, a sc injection of either KP-10 into female (OVX+E+P) mice or GnRH into (OVX+E) mice failed to stimulate lordosis behavior in nNOS^{-/-} females (Tukey's multiple comparison test; respectively P=0.56; P=0.19; FIG. 11c). In a final experiment, Kiss^{-/-} (OVX+E+P) females were injected sc with the cocktail SNAP+BAY 41-2272 and WT-like levels of lordosis behavior were observed (Paired t test; P=0.02; FIG. 11d).

Taken together, these data demonstrate that NO is a key neurotransmitter downstream of kisspeptin neurons mediating both mate preference and lordosis behavior. The nNOS neuron population in the VMHv1³⁵ might be a potential important downstream relay of RP3V kisspeptin neurons in governing lordosis behavior but also mate preference.

### REFERENCES

1. Boling J L, Blandau R J. The estrogen-progesterone induction of mating responses in the spayed female rat. Endocrinology 25, 359-364 (1939).
2. McClintock M K. Estrous synchrony: modulation of ovarian cycle length by female pheromones. Physiology & behavior 32, 701-705 (1984).
3. Brown R E. Mammalian social odors: a critical review. Advances in the Study of Behavior 10, 103-162 (1979).
4. Brennan P A, Zufall F. Pheromonal communication in vertebrates. Nature 444, 308-315 (2006).
5. Bruce H M. An exteroceptive block to pregnancy in the mouse. Nature 184, 105 (1959).
6. Halpern M, Martinez-Marcos A. Structure and function of the vomeronasal system: an update. Prog Neurobiol 70, 245-318 (2003).
7. Vandenbergh J G, Whitsett J M, Lombardi J R. Partial isolation of a pheromone accelerating puberty in female mice. Journal of reproduction and fertility 43, 515-523 (1975).
8. Whitten W K, Bronson F H, Greenstein J A. Estrus-inducing pheromone of male mice: transport by movement of air. Science 161, 584-585 (1968).
9. Boehm U, Zou Z, Buck L B. Feedback loops link odor and pheromone signaling with reproduction. Cell 123, 683-695 (2005).
10. Herbison A E. Physiology of the gonadotropin-releasing hormone neuronal network (2006).
11. Bronson F H, Maruniak J A. Differential effects of male stimuli on follicle-stimulating hormone, luteinizing hormone, and prolactin secretion in prepubertal female mice. Endocrinology 98, 1101-1108 (1976).
12. Yoon H, Enquist L W, Dulac C. Olfactory inputs to hypothalamic neurons controlling reproduction and fertility. Cell 123, 669-682 (2005).
13. Bronson F H, Desjardins C. Endocrine responses to sexual arousal in male mice. Endocrinology 111, 1286-1291 (1982).
14. Han S K, et al. Activation of gonadotropin-releasing hormone neurons by kisspeptin as a neuroendocrine switch for the onset of puberty. The Journal of neuroscience: the official journal of the Society for Neuroscience 25, 11349-11356 (2005).
15. Irwig M S, et al. Kisspeptin activation of gonadotropin releasing hormone neurons and regulation of KiSS-1 mRNA in the male rat. Neuroendocrinology 80, 264-272 (2004).
16. Hu M H, et al. Relative Importance of the Arcuate and Anteroventral Periventricular Kisspeptin Neurons in Control of Puberty and Reproductive Function in Female Rats. Endocrinology 156, 2619-2631 (2015).
17. Smith J T, Popa S M, Clifton D K, Hoffman G E, Steiner R A. Kiss1 neurons in the forebrain as central processors for generating the preovulatory luteinizing hormone surge. The Journal of neuroscience: the official journal of the Society for Neuroscience 26, 6687-6694 (2006).
18. Clarkson J, Herbison A E. Postnatal development of kisspeptin neurons in mouse hypothalamus; sexual dimorphism and projections to gonadotropin-releasing hormone neurons. Endocrinology 147, 5817-5825 (2006).
19. Bakker J, Pierman S, Gonzalez-Martinez D. Effects of aromatase mutation (ArKO) on the sexual differentiation of kisspeptin neuronal numbers and their activation by same versus opposite sex urinary pheromones. Hormones and behavior 57, 390-395 (2010).
20. Bakker J. Sexual differentiation of the neuroendocrine mechanisms regulating mate recognition in mammals. Journal of neuroendocrinology 15, 615-621 (2003).
21. Keller M, Pierman S, Douhard Q, Baum M J, Bakker J. The vomeronasal organ is required for the expression of lordosis behaviour, but not sex discrimination in female mice. Eur J Neurosci 23, 521-530 (2006).
22. d'Anglemont de Tassigny X, et al. Hypogonadotropic hypogonadism in mice lacking a functional Kiss1 gene. Proceedings of the National Academy of Sciences of the United States of America 104, 10714-10719 (2007).
23. Yang C F, et al. Sexually dimorphic neurons in the ventromedial hypothalamus govern mating in both sexes and aggression in males. Cell 153, 896-909 (2013).
24. Mayer C, et al. Timing and completion of puberty in female mice depend on estrogen receptor alpha-signaling in kisspeptin neurons. Proceedings of the National Academy of Sciences of the United States of America 107, 22693-22698 (2010).
25. Liu X, et al. Frequency-dependent recruitment of fast amino acid and slow neuropeptide neurotransmitter release controls gonadotropin-releasing hormone neuron excitability. The Journal of neuroscience: the official journal of the Society for Neuroscience 31, 2421-2430 (2011).
26. Yip S H, Boehm U, Herbison A E, Campbell R E. Conditional Viral Tract Tracing Delineates the Projections of the Distinct Kisspeptin Neuron Populations to Gonadotropin-Releasing Hormone (GnRH) Neurons in the Mouse. Endocrinology 156, 2582-2594 (2015).
27. Dubois S L, et al. Positive, but not negative feedback actions of estradiol in adult female mice require estrogen receptor alpha in kisspeptin neurons. Endocrinology 156, 1111-1120 (2015).
28. Piet R, Boehm U, Herbison A E. Estrous cycle plasticity in the hyperpolarization-activated current ih is mediated by circulating 17beta-estradiol in preoptic area kisspeptin neurons. The Journal of neuroscience: the official journal of the Society for Neuroscience 33, 10828-10839 (2013).
29. Mayer C, Boehm U. Female reproductive maturation in the absence of kisspeptin/GPR54 signaling. Nature neuroscience 14, 704-710 (2011).
30. Messina A, et al. A microRNA switch regulates the rise in hypothalamic GnRH production before puberty. Nature neuroscience 19, 835-844 (2016).
31. Kumar D, Freese M, Drexler D, Hermans-Borgmeyer I, Marquardt A, Boehm U. Murine arcuate nucleus kisspeptin neurons communicate with GnRH neurons in utero. The Journal of neuroscience: the official journal of the Society for Neuroscience 34, 3756-3766 (2014).
32. Pfaff D W, Sakuma Y. Facilitation of the lordosis reflex of female rats from the ventromedial nucleus of the hypothalamus. J Physiol 288, 189-202 (1979).
33. Gonzalez-Flores O, Etgen A M. The nitric oxide pathway participates in estrous behavior induced by progesterone and some of its ring A-reduced metabolites. Hormones and behavior 45, 50-57 (2004).
34. Mani S K, Allen J M, Rettori V, McCann S M, O'Malley B W, Clark J H. Nitric oxide mediates sexual behavior in female rats. Proceedings of the National Academy of Sciences of the United States of America 91, 6468-6472 (1994).
35. Lee H, et al. Scalable control of mounting and attack by Esr1+ neurons in the ventromedial hypothalamus. Nature 509, 627-632 (2014).
36. Dudley C A, Moss R L. Facilitation of lordosis in female rats by CNS-site specific infusions of an LH-RH fragment, Ac-LH-RH-(5-10). Brain Res 441, 161-167 (1988).
37. Foreman M M, Moss R L. Effects of subcutaneous injection and intrahypothalamic infusion of releasing hormones upon lordotic response to repetitive coital stimulation. Hormones and behavior 8, 219-234 (1977).
38. Moss R L, McCann S M. Induction of mating behavior in rats by luteinizing hormone-releasing factor. Science 181, 177-179 (1973).
39. Pfaff D W. Luteinizing hormone-releasing factor potentiates lordosis behavior in hypophysectomized ovariectomized female rats. Science 182, 1148-1149 (1973).
40. Ward B J, Charlton H M. Female sexual behaviour in the GnRH deficient, hypogonadal (hpg) mouse. Physiology & behavior 27, 1107-1109 (1981).
41. Kauffman A S, et al. The kisspeptin receptor GPR54 is required for sexual differentiation of the brain and behavior. The Journal of neuroscience: the official journal of the Society for Neuroscience 27, 8826-8835 (2007).
42. Lyubimov Y, Engstrom M, Wurster S, Savola J M, Korpi E R, Panula P. Human kisspeptins activate neuropeptide FF2 receptor. Neuroscience 170, 117-122 (2010).
43. Oishi S, et al. Activation of Neuropeptide F F Receptors by Kisspeptin Receptor Ligands. ACS Med Chem Lett 2, 53-57 (2011).
44. Liu X, Herbison A. Kisspeptin regulation of arcuate neuron excitability in kisspeptin receptor knockout mice. Endocrinology 156, 1815-1827 (2015).
45. Gouarderes C, Puget A, Zajac J M. Detailed distribution of neuropeptide F F receptors (NPFF1 and NPFF2) in the rat, mouse, octodon, rabbit, guinea pig, and marmoset monkey brains: a comparative autoradiographic study. Synapse 51, 249-269 (2004).
46. Kumar D, Candlish M, Periasamy V, Avcu N, Mayer C, Boehm U. Specialized subpopulations of kisspeptin neurons communicate with GnRH neurons in female mice. Endocrinology 156, 32-38 (2015).
47. Tabuchi K, et al. GAL4/UAS-WGA system as a powerful tool for tracing Drosophila transsynaptic neural pathways. J Neurosci Res 59, 94-99 (2000).
48. Dey S, et al. Cyclic Regulation of Sensory Perception by a Female Hormone Alters Behavior. Cell 161, 1334-1344 (2015).
49. MacLusky N J, McEwen B S. Oestrogen modulates progestin receptor concentrations in some rat brain regions but not in others. Nature 274, 276-278 (1978).
50. Thornton J E, Nock B, McEwen B S, Feder H H. Estrogen induction of progestin receptors in microdissected hypothalamic and limbic nuclei of female guinea pigs. Neuroendocrinology 43, 182-188 (1986).
51. Auger A P, Blaustein J D. Progesterone enhances an estradiol-induced increase in Fos immunoreactivity in localized regions of female rat forebrain. The Journal of neuroscience: the official journal of the Society for Neuroscience 15, 2272-2279 (1995).
52. Brock O, Douhard Q, Baum M J, Bakker J. Reduced prepubertal expression of progesterone receptor in the hypothalamus of female aromatase knockout mice. Endocrinology 151, 1814-1821 (2010).
53. Chachlaki K, et al. Phenotyping of nNOS neurons in the postnatal and adult female mouse hypothalamus. The Journal of comparative neurology 525, 3177-3189 (2017).
54. Hanchate N K, et al. Kisspeptin-GPR54 signaling in mouse NO-synthesizing neurons participates in the hypothalamic control of ovulation. The Journal of neuroscience: the official journal of the Society for Neuroscience 32, 932-945 (2012).
55. Argiolas A, Melis M R. Neuropeptides and central control of sexual behaviour from the past to the present: a review. Prog Neurobiol 108, 80-107 (2013).
56. Becker R O, et al. Sexual behavior and dendritic spine density of posterodorsal medial amygdala neurons in oxytocin knockout female mice. Behavioural brain research 256, 95-100 (2013).
57. Nappi R E, et al. Menopause and sexual desire: the role of testosterone. Menopause Int 16, 162-168 (2010).
58. Comninos A N, et al. Kisspeptin modulates sexual and emotional brain processing in humans. The Journal of clinical investigation 127, 709-719 (2017).
59. Cardin J A, et al. Driving fast-spiking cells induces gamma rhythm and controls sensory responses. Nature 459, 663-667 (2009).
60. Harfe B D, McManus M T, Mansfield J H, Hornstein E, Tabin C J. The RNaselll enzyme Dicer is required for morphogenesis but not patterning of the vertebrate limb. Proceedings of the National Academy of Sciences of the United States of America 102, 10898-10903 (2005).
61. Huang P L, Dawson T M, Bredt D S, Snyder S H, Fishman M C. Targeted disruption of the neuronal nitric oxide synthase gene. Cell 75, 1273-1286 (1993).
62. Bakker J, Honda S, Harada N, Balthazart J. The aromatase knock-out mouse provides new evidence that estradiol is required during development in the female for the expression of sociosexual behaviors in adulthood. The Journal of neuroscience: the official journal of the Society for Neuroscience 22, 9104-9112 (2002).
63. Paxinos G, Franklin K B J. The Mouse Brain in Stereotaxic Coordinates. Academic Press (2001).
64. Bronson F H. The regulation of luteinizing hormone secretion by estrogen: relationships among negative feedback, surge potential, and male stimulation in juvenile, peripubertal, and adult female mice. Endocrinology 108, 506-516 (1981).
65. Wysocki C J, Wysocki L. Experimental Cell Biology of Taste and Olfaction. CRC Press (1995).
66. Martel K L, Baum M J. Adult testosterone treatment but not surgical disruption of vomeronasal function augments male-typical sexual behavior in female mice. The Journal of neuroscience: the official journal of the Society for Neuroscience 29, 7658-7666 (2009).
67. Brock O, Bakker J, Baum M J. Assessment of urinary pheromone discrimination, partner preference, and mating behaviors in female mice. Methods Mol Biol 1068, 319-329 (2013).
68. Ebbesson S O E. Contemporary research methods in neuroanatomy. Springer-Verlag (1970).
69. Brock O, Bakker J. The two kisspeptin neuronal populations are differentially organized and activated by estradiol in mice. Endocrinology 154, 2739-2749 (2013).
70. Boillat M, Challet L, Rossier D, Kan C, Carleton A, Rodriguez I. The vomeronasal system mediates sick conspecific avoidance. Current biology: CB 25, 251-255 (2015).

### Example 2. Subcutaneous Injection of Kisspeptin-54 in Human Female Patients Diagnosed with HSDD Increases Sexual Desire in Said Patients

20 human female patients diagnosed with HSDD receive either a single subcutaneous (sc) injection of kisspeptin-54 (SEQ ID NO: 17) at a dose of 6.4 nmol/kg of body weight or a sc injection of a saline (i.e. control). The sexual desire of the patients is measured by determining their brain activation using magnetic resonance imaging (MRI) with the blood-oxygenation-level-dependent (BOLD) technique to identify and quantify brain regions associated with visually evoked sexual arousal in women. All female patients are subjected to two sessions on separate days with at least 1 month in between the sessions to act as their own control. They receive in one session kisspeptin-54 and in the other one saline, the order will be randomized. Female patients are positioned into the MRI scanner 30 minutes after the sc injection and three dimensional images are taken of the brain when watching a series of erotic pictures as well as more neutral pictures to serve as control condition using an event-related design. The task is followed by an anatomical scan in which high resolution T1-weighted images are taken to determine the anatomical boundaries of the activation observed during the visual task. The human female patients who are administered kisspeptin-54 show increased brain activation when watching the images in comparison to when they do not receive saline.

### Example 3: Subcutaneous Injection of Kisspeptin-54 in Human Female Patients Diagnosed with HSDD Increases Sexual Desire in Said Patients

20 human female patients diagnosed with HSDD receive either a single subcutaneous (sc) injection of kisspeptin-54 (SEQ ID NO: 17) at a dose of 6.4 nmol/kg of body weight or a sc injection of saline (i.e. control). The sexual desire of the patients is measured using vaginal photoplethysmography, which has been used to assess female genital arousal. It consists of a tampon-sized acrylic device containing a light emitting diode and a phototransistor to detect light to measure vaginal blood flow with increased sexual desire leading to increased vaginal blood flow. The sexual desire of the patients is scored starting 15 minutes after administration of sc kisspeptin or saline when watching either erotic images or neutral images. Female HSDD patients taking kisspeptin show increased vaginal blood flow when watching erotic images but not when watching neutral images or when receiving saline. Any effects of kisspeptin on sexual desire disappear by 4 h after the injection.

1. An agonist of a human kisspeptin receptor for use in a method of treating a hypoactive sexual desire disorder (HSDD) in human females; wherein the agonist is human kisspeptin or a biologically active fragment or variant of human kisspeptin, or a pharmaceutically acceptable salt thereof.

2. The agonist for use according to claim 1, wherein said fragment comprises or consists of the amino acid sequence YNWNSFGLRF (SEQ ID NO: 20).

3. The agonist for use according to claim 1, wherein said variant displays at least 80% overall amino acid sequence identity to wild-type human kisspeptin or fragment thereof.

4. The agonist for use according to claim 1, wherein said variant comprises one or more non-naturally occurring amino acids, chemically modified amino acids and/or D-amino acids.

5. The agonist for use according to claim 1, wherein the agonist is selected from the group consisting of human kisspeptin-54, human kisspeptin-14, human kisspeptin-13, human kisspeptin-10, pharmaceutically acceptable salts thereof, and combinations thereof.

6. The agonist for use according to claim 1, wherein the agonist is human kisspeptin-10 or a pharmaceutically acceptable salt thereof.

7. A method of treating HSDD in a human female patient, comprising administering to said patient a therapeutically effective amount of an agonist of a human kisspeptin receptor; wherein the agonist is human kisspeptin or a biologically active fragment or variant of human kisspeptin, or a pharmaceutically acceptable salt thereof.

8. The method according to claim 7, wherein the agonist:
comprises or consists of the amino acid sequence YNWNSFGLRF (SEQ ID NO: 20);
is a variant of human kisspeptin having at least 80% overall amino acid sequence identity to wild-type human kisspeptin or fragment thereof;
is a variant of human kisspeptin and comprises one or more non-naturally occurring amino acids, chemically modified amino acids, D-amino acids, or a combination thereof;
is selected from the group consisting of human kisspeptin-54, human kisspeptin-14, human kisspeptin-13, human kisspeptin-10, pharmaceutically acceptable salts thereof, and combinations thereof, or
is human kisspeptin-10 or a pharmaceutically acceptable salt thereof.

9. The method according to claim 7, wherein the HSDD comprises absence of sexual desire, loss of sexual desire, or decrease in sexual desire.

10. A non-therapeutic method of enhancing libido in a human female subject, comprising administering to said subject an amount of an agonist of a human kisspeptin receptor sufficient to enhance libido in said subject; wherein the agonist is human kisspeptin or a biologically active fragment or variant of human kisspeptin, or a pharmaceutically acceptable salt thereof.

11. A non-therapeutic method of inducing sexual arousal in a human female subject, comprising administering to said subject an amount of an agonist of a human kisspeptin receptor sufficient to induce sexual arousal in said subject; wherein the agonist is human kisspeptin or a biologically active fragment or variant of human kisspeptin, or a pharmaceutically acceptable salt thereof.

12. The method of claim 10, wherein the agonist:
comprises or consists of the amino acid sequence YNWNSFGLRF (SEQ ID NO: 20);
is a variant of human kisspeptin having at least 80% overall amino acid sequence identity to wild-type human kisspeptin or fragment thereof;
is a variant of human kisspeptin and comprises one or more non-naturally occurring amino acids, chemically modified amino acids, D-amino acids, or a combination thereof;
is selected from the group consisting of human kisspeptin-54, human kisspeptin-14, human kisspeptin-13, human kisspeptin-10, pharmaceutically acceptable salts thereof, and combinations thereof, or
is human kisspeptin-10 or a pharmaceutically acceptable salt thereof.

13. The method according to claim 11, wherein the agonist is administered prior to sexual activity.

14. The method according to claim 7, wherein the agonist is administered at a molar amount between 0.1 and 10 nmol per kg body weight.

15. The method according to claim 7, wherein the agonist is administered intranasally, transdermally, orally, intravenously or subcutaneously.

16. The method according to claim 15, wherein the agonist is administered by a transdermal patch, a nasal spray, intravenous bolus injection, subcutaneous bolus injection, or intravenous infusion.

17. The method according to claim 7, wherein the agonist is administered in combination with one or more other therapeutic suitable for treating the disorder of sexual desire in the human female.

18. The method according to claim 17, wherein the one or more other therapeutic is selected from the group consisting of androstadienone, flibanserin, testosterone, prasterone, trazodone, bremelanotide, bupropion, buspirone, sildenafil, lasofoxifene, BP-101, PL-6983, TGFK09SD, and combinations thereof.

19. The method according to claim 17, wherein the one or more other therapeutic is selected from the group consisting of the combination of bupropion and trazodone, the combination of buspirone and testosterone, and the combination of sildenafil and testosterone.

20. (canceled)

21. (canceled)


SEQUENCE LISTING (30 sequences)

SEQ ID NO: 1 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELLRLGSHPAPARAQKPGSSGLAARGLCVLGEDNAPL

SEQ ID NO: 2 - protein, 46 aa, Homo sapiens
MHTVATSGPNASWGAPANASGCPGCGANASDGPVPSPRAVDAWLVP

SEQ ID NO: 3 - protein, 19 aa, Homo sapiens
LYPLPGWVLGDFMCKFVNY

SEQ ID NO: 4 - protein, 27 aa, Homo sapiens
ALHRLSPGPRAYCSEAFPSRALERAFA

SEQ ID NO: 5 - protein, 21 aa, Homo sapiens
LQALGPAGSWHPRSYAAYALK

SEQ ID NO: 6 - protein, 43 aa, Homo sapiens
MHTVATSGPNASWGAPANASGCPGCGANASDGPVPSPRAVDAW

SEQ ID NO: 7 - protein, 18 aa, Homo sapiens
LYPLPGWVLGDFMCKFVN

SEQ ID NO: 8 - protein, 25 aa, Homo sapiens
HRLSPGPRAYCSEAFPSRALERAFA

SEQ ID NO: 9 - protein, 18 aa, Homo sapiens
ALGPAGSWHPRSYAAYAL

SEQ ID NO: 10 - protein, 46 aa, Homo sapiens; identical to SEQ ID NO: 2

SEQ ID NO: 11 - protein, 19 aa, Homo sapiens
LYPLPGWVLGDFMCKFVNY

SEQ ID NO: 12 - protein, 24 aa, Homo sapiens
ALHRLSPGPRAYCSEAFPSRALER

SEQ ID NO: 13 - protein, 21 aa, Homo sapiens
LQALGPAGSWHPRSYAAYALK

SEQ ID NO: 14 - protein, 138 aa, Homo sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 15 - protein, 19 aa, Homo sapiens
MNSLVSWQLLLFLCATHFG

SEQ ID NO: 16 - protein, 119 aa, Homo sapiens
EPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 17 - protein, 54 aa, Homo sapiens
GTSLSPPPESSGSPQQPGLSAPHSRQIPAPQGAVLVQREKDLPNYNWNSFGLRF

SEQ ID NO: 18 - protein, 14 aa, Homo sapiens
DLPNYNWNSFGLRF

SEQ ID NO: 19 - protein, 13 aa, Homo sapiens
LPNYNWNSFGLRF

SEQ ID NO: 20 - protein, 10 aa, Homo sapiens
YNWNSFGLRF

SEQ ID NO: 21 - protein, 16 aa, Homo sapiens
LPNYNWNSFGLRFGKR

SEQ ID NO: 22 - protein, 13 aa, Homo sapiens
YNWNSFGLRFGKR

SEQ ID NO: 23 - protein, 14 aa, Homo sapiens
PNYNWNSFGLRFGK

SEQ ID NO: 24 - protein, 12 aa, Homo sapiens
PNYNWNSFGLRF

SEQ ID NO: 25 - protein, 12 aa, Homo sapiens
YNWNSFGLRFGK

SEQ ID NO: 26 - protein, 12 aa, Homo sapiens
NYNWNSFGLRFG

SEQ ID NO: 27 - protein, 11 aa, Homo sapiens
NYNWNSFGLRF

SEQ ID NO: 28 - protein, 11 aa, Homo sapiens
YNWNSFGLRFG

SEQ ID NO: 29 - protein, 9 aa, Homo sapiens
NWNSFGLRF

SEQ ID NO: 30 - protein, 9 aa, Homo sapiens
YNWNSFGLR